(19) United States
(12) Patent Application Publication
(10) Pub. No.: US 2020/0216492 A1
(43) Pub. Date: Jul. 9, 2020

(54) USE OF C-TERMINALLY EXTENDED PEPTIDES TO DISRUPT INHIBITOR NK CELL RECEPTOR INTERACTIONS WITH MHC I

(71) Applicant: La Jolla Institute for Allergy and Immunology, La Jolla, CA (US)

(72) Inventors: Dirk ZAJONC, San Diego, CA (US); Bjoern PETERS, San Diego, CA (US); Soumya Govinda REMESH, Berkeley, CA (US); Ge YING, San Diego, CA (US)

(21) Appl. No.: 16/348,839

(22) PCT Filed: Nov. 9, 2017
(86) PCT No.: PCT/US2017/060901

§ 371 Date: May 9, 2019

Related U.S. Application Data
(60) Provisional application No. 62/419,882, filed on Nov. 9, 2016.

(51) Int. Cl. C07K 7/04; C07K 14/47; C07K 14/74; A61P 35/00

(52) U.S. Cl. CPC C07K 7/04; C07K 14/4748; A61K 38/00; A61P 35/00; C07K 14/70539

(57) ABSTRACT

Presented herein, in certain embodiments, are compositions comprising synthetic polypeptides that specifically bind to MHC Class I.

### RELATED APPLICATIONS

This patent application claims the benefit of U.S. patent application No. 62/419,882 filed on Nov. 9, 2016, which is expressly incorporated herein by reference in its entirety.

### FIELD OF THE INVENTION

Embodiments of the invention relate to synthetic polypeptides that bind MHC Class I molecules and uses thereof.

### INTRODUCTION

Presented herein are novel short synthetic polypeptides that bind MHC Class I molecules, compounds comprising synthetic polypeptides that bind MHC Class I molecules, pharmaceutical compositions thereof and methods of using the same.

### SUMMARY

In some aspects, presented herein is a synthetic polypeptide comprising a first peptide sequence consisting of 7 to 11 amino acids in length and comprising an MHC class I binding epitope, and no more than one additional amino acid covalently linked to a C-terminus of the first peptide, wherein the one additional amino acid comprises an electronegative or electropositive charged side group, or a second peptide consisting of 2 to 7 amino acids in length covalently linked to a C-terminus of the first peptide, wherein the second peptide comprises at least one electronegative or electropositive charged side group. The MHC class I binding epitope may be an HLA-A, HLA-B or HLA-C epitope. In some embodiments, the additional amino acid or second peptide sequence is covalently linked to the C-terminus of the first amino acid sequence by a peptide bond.

In some aspects, presented herein is method of treating a subject having a neoplasia, neoplastic disorder or cancer comprising providing a subject having, or suspected of having, a neoplastic disorder; and administering a therapeutically effective amount of a synthetic polypeptide described herein to the subject. In certain embodiments, a method of treating a subject having a neoplasia, neoplastic disorder or cancer comprises administering a therapeutically effective amount of the macromolecule, compound or composition to the subject, where the macromolecule, compound or composition comprises at least one synthetic polypeptide described herein. In certain embodiments, a neoplastic disorder or cancer comprises a carcinoma, neuroblastoma, hepatocellular cancer, sarcoma, lymphoma, leukemia, mesothelioma, glioblastoma, myeloma, melanoma.

In some embodiments, a method comprises contacting a cell, tumor, cancer or malignant cell, with a synthetic polypeptide described herein or with a macromolecule, compound or composition described herein where the macromolecule, compound or composition comprises at least one synthetic polypeptide described herein.

In certain embodiments, a method comprises reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprising administering to the subject an amount of a synthetic polypeptide described herein sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

In some aspects, presented herein is a method of treating a subject having an infectious disease, or suspected of having an infectious disease, the method comprising administering a therapeutically effective amount of the synthetic polypeptide to the subject, or administering a macromolecule, compound or composition to the subject where the macromolecule, compound or composition described herein where the macromolecule, compound or composition comprises at least one synthetic polypeptide described herein. An infectious disease may comprise a virus infection, a bacteria infection or a parasite infection.

In some embodiments, a pharmaceutical composition comprises a synthetic polypeptide. In some embodiments, a pharmaceutical composition comprises a pharmaceutically acceptable excipient, diluent, additive and/or carrier. In some embodiments, a pharmaceutical composition is formulated as a sterile, lyophilized powder, that upon reconstitution, is suitable for intravenous administration to a subject.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1. HLA-A*02:01-peptide structures. FIG. 1 shows an overall structure of HLA-A*02:01 with heavy chain in gray and light chain (β2M) in blue. Peptide backbones are superimposed within the binding grove.

FIGS. 2A-C, FIGS. 3A & B and FIGS. 4A & B show molecular surface representation of the binding grove of HLA-A*02:01 with the individual peptides bound. Peptide sequences are labeled and charged amino acids are colored in Blue (positive) and red (negative). 2FoFc Electron density for the peptide (shown in blue mesh) is contoured at 1σ.

FIGS. 5 A-C show detailed hydrogen bond interactions around the F pocket. HLA-A*02:01 is shown in grey, peptides in color and electron density for Lys146 as a blue mesh contoured at 1σ. Hydrogen bonds between 2.5-3.65 Å are shown as blue dashed lines. Structures of nested peptides are shown in the top panel and the corresponding extending peptides below. FIG. 5A Peptide pair GLKEGIPAL (green) and GLKEGIPALD (cyan); FIG. 5B GLLPELPAV (pink) and GLLPELPAVGGNE (grey); FIG. 5C peptide pair YLSPIASPL (violet) and YLSPIASPLLDGKSLR (orange).

FIG. 6 shows HLA-peptide TDF assay. First derivative of melt curve is shown. Longer peptides have a 10° reduced Tm but form stable complexes with MHC I.

FIGS. 7 A-D show a binding comparison between nested and extending peptides. A) Binding grove of HLA-A*02:01 with the nested peptide GLKEGIPAL (green) superimposed with extended peptide GLKEGIPALD (cyan). B) Superimposition of nested peptide GLLPELPAV (pink) with extended peptide GLLPELPAVGGNE (grey). C) Superimposition of nested peptide YLSPIASPL (violet) with extended peptide YLSPIASPLLDGKSLR (orange). D) Superimposition of nested peptide YLSPIASPL (violet) with nested peptide YLSPIASPLL (yellow).

FIGS. 8A-C show a mechanism of F pocket opening and peptide sequences. A) ‘Tyrosine swing’: Y84 adopts a different rotamer to open the F pocket and accommodate longer peptide FVLELEPEWTVK (UFP¹⁶⁻²⁷) (15). B) ‘Lysine lift’: Lifting of residue K146 to accommodate longer peptides including GLKEGIPALD, GLLPELPAVGGNE and YLSPIASPLLDGKSLR. C) List of nested and longer T. gondii peptides. Positively charged residues (blue) open the F pocket via the ‘Tyrosine swing’, while negatively charged residues (red) open the biding grove via the ‘Lysine lift’.

FIGS. 9A-B show a mechanism of NK cell activation and MHC I binding sites for NK receptors. (A) Activation of NK cells is determined by the sum of inhibitory and activating signals. (B) Binding sites for different activating and inhibitory NK receptors on MHC I. KIR2DL bind above the F pocket of MHC I.

FIG. 10 shows KIR classification and ligand specificities. KIR are highly polymorphic receptors and engage a variety of HLA ligands.

FIGS. 11A-B show HLA-peptide TDF assay. Longer peptides have a 10° reduced Tm but form stable complexes with MHC I.

FIGS. 12A-D show core and extended peptide binding to HLA-A*02:01. (A) core peptide F11V binds to HLA in a zig-zag conformation, while extended peptide F12K opens the F pocket to allow the additional lysine to protrude into the solvent (B). Peptide binding is very well ordered, judged by the well-defined electron density (C, D).

FIGS. 13A-B show F pocket opening mechanism. Y84 swing and K146 lift is determined by the first charged residue following the core peptide. Structures containing core peptides in green, extending peptides in yellow and cyan. In orange HLA-A*02:01 with calreticulin decamer peptide MLLSVPLLLG.

FIG. 14 shows variability of HLA-A, -B, and -C. Residues Tyr84 and Lys146 are conserved across all HLA alleles, while residue 80 is more variable. Height of red bar indicates level of sequence variability for any given amino acid.

FIG. 15 shows refolding of HLA-B and HLA-C alleles. Successful refolding of HLA-B*57:01 with indicated peptides is demonstrated by proper migration during SEC and on SDS-PAGE (not shown). HLA-C*03:07 does not show a distinct peak in comparison to the control peptide G9L and no associated peptide could be detected by MALDI-TOF (not shown). HLA-C*04:01 refolding reveals a clearly separated peak at the expected elution volume and MALDI-TOF verified presence of extended peptides (not shown). Note that A280 scale is different between the three panels.

FIG. 16 shows amino acid variations around the F pocket. While Y84 and K146 are conserved across all alleles, amino acids at position 80 and 142 can differ. Peptides shown as a simple tube.

FIG. 17 shows Refolding of HLA-A*02:01 mutants with extending peptide G11N and F12K. Refolding efficiency is judged by comparing peak high (at 45 kDa) with that of the preceding oligomeric peak. MW standard indicated as a grey line.

FIG. 18 shows crystal structure of HLA-C*04:01-Y9F. Electron density (2FoFc at 1σ) shown as blue mesh around peptide Y9F (yellow sticks). HLA in grey with F pocket lining residues shown as sticks.

FIG. 19 shows KIR2DL1-Fc binding to HLA-C*04:01 is abrogated by C-terminally extending peptides Y10K and Y1 N. Indicated HLA-peptide complexes were immobilized on streptavidin sensor tips and binding to a 2 μM solution of KIR2DL1-Fc was measured in real-time by BLI (Octet RED96, Forte Bio). Binding to buffer only was used for background subtraction.

FIG. 20 shows tetramer staining of KIR3DL1 transfected YTS cells. KIR3DL1 negative (top) and positive (bottom) YTS cells were analyzed for their staining using the indicated HLA-B/peptide tetramer. Binding of the tetramer to KIR3DL1 negative cells likely indicates binding of the tetramer to another receptor, such as 2B4 and, therefore, different KIR transfected cells, such as K562 cells will be used next.

FIG. 21 shows cytotoxicity assay using YTS cells expressing KIR3DL1 and KIR2DL1. HLA-C*04:01 (Cw4) cells tolerize KIR2DL1-YTS cells but are killed by KIR3DL1 expressing YTS cells (top). Addition of peptide L12K appears to induce killing of HLA-B*58:02 transfected 721.221 cells by blocking KIR3DL1-HLA-B interaction (bottom).

FIG. 22 shows an example of antigen processing and (cross-) presentation.

FIG. 23 shows MHC I binds peptides “tucked in”.

FIG. 24 shows HLA-A*02:01 peptides.

FIG. 25 shows K146 opens the F′ pocket.

FIG. 26 shows KIR family of NK receptors and ligands.

### DETAILED DESCRIPTION

Peptide antigen-presentation by Major Histocompatibility Class (MHC) I proteins initiates CD8 T cell mediated immunity against pathogens and cancers. MHC I molecules typically bind peptides with nine amino acids in length with both ends tucked inside the major A and F binding pocket. Longer peptides either bulge out of the groove in the middle of the peptide or bind in a zig-zag fashion inside the groove. The present invention establishes an alternative binding mode, induced by naturally occurring peptides from Toxoplasma gondii that were bound by HLA-A2*02:01. These peptides were extended at the C-terminus and contained charged residues after the anchor amino acid at P9, which enabled them to open the F pocket and extend their C-terminal extension into the solvent. The mechanism of F pocket opening is dictated, in part, by the charge of the first charged amino acid found within the extension. While positively charged amino acid result in a Tyr84 swing, amino acids that are negatively charged induce a Lys146 lift. Presented herein, in some embodiments, are synthetic peptides that bind MHC Class I molecules, or a portion thereof, as well as compositions and uses thereof. The novel synthetic peptides presented herein comprise a C-terminal extension comprising up to 7 additional amino acids, where at least one is a charged amino acid. These synthetic peptides are longer than typical MHC Class I binding peptides and upon binding MHC Class I molecules, the C-terminal end is extended into the solvent thereby altering interaction of the peptide-bound MHC Class I complex with NK cells. The altered interaction can result in the activation of NK cells and induce NK cell killing of a cell (e.g., a cancer cell, a lymphoma cell, a virus infected cell) that presents the peptide-bound MHC Class I complex.

The term “subject” refers to a mammal. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a human has or is suspected of having a cancer or neoplastic disorder.

In some embodiments a subject is in need of a treatment or composition described herein. In certain embodiments a subject has or is suspected of having a neoplastic disorder, neoplasia, tumor, malignancy or cancer. In some embodiments a subject in need of a treatment or composition described herein has or is suspected of having a neoplastic disorder, neoplasia, tumor, malignancy or cancer. In certain embodiments a synthetic polypeptide or composition described herein is used to treat a subject having, or suspected of having, a neoplastic disorder, neoplasia, tumor, malignancy or cancer.

Presented herein are novel synthetic polypeptides (referred to herein simply as “synthetic polypeptides”) that bind major histocompatibility complex (MHC) Class I molecules, non-limiting examples of which include polymorphic variations of HLA-A, HLA-B, HLA-C, HLA-E, HLA-F and HLA-G. Class I MHC molecules bind peptides generated mainly from degradation of cytosolic proteins by the proteasome. It is generally understood that the MHC I: peptide complex is then inserted via endoplasmic reticulum into the external plasma membrane of the cell. The epitope peptide is typically bound on an extracellular portion of the class I MHC molecule for presentation to another cell. Thus, the function of the class I MHC is to display self-antigen or foreign antigens, in the form of short peptides, to cytotoxic T lymphocytes (CTLs), natural killer (NK) cells, and certain other cell types. A normal (e.g., healthy, non-infected, non-cancerous, or non-malignant) cell will typically display peptides from normal cellular protein turnover on its class I MHC. CTLs and NK cells that subsequently engage the MHC class I/self-antigen complex typically will not be activated in response to the self-antigens presented. When a cell expresses foreign antigens presented in the form of an MHC Class I/peptide complex, CTLs and/or NK cells specific for the MHC/peptide complex will often kill the presenting cell.

In certain embodiments, a synthetic polypeptide comprise to a first portion, or first peptide comprising an MHC class I binding peptide and a second portion, or second peptide comprising 2-7 amino acids. In certain embodiments, a synthetic polypeptide comprise to a first portion, or first peptide comprising an MHC class I binding peptide and a second portion consisting of one additional amino acid. The first peptide is typically covalently linked to the second peptide or additional amino acid. In certain embodiments, the first peptide is covalently linked to the second peptide by a peptide bond. In certain embodiments, the C-terminus (i.e. carboxy terminus) of the first peptide is covalently linked to the N-terminus of the second peptide by a peptide bond. The second peptide generally represents an extension of an MHC class I binding peptide or MHC class I epitope. In certain embodiments, the amino acid sequence of a first peptide is 7 to 11 amino acids in length. In certain embodiments, the amino acid sequence of a first peptide is 7, 8, 9, 10, or 11 amino acids in length.

In some embodiments, the first peptide of a synthetic polypeptide comprises or consists of an MHC class I epitope. An MHC class I epitope can be an HLA-A, HLA-B, HLA-C, HLA-E, HLA-F or HLA-G epitope. An MHC class I epitope can be any suitable MHC class I epitope that is known, identified or predicted. Accordingly, in certain embodiments, a synthetic polypeptide comprises a first peptide, wherein the first peptide comprises or consists of an amino acid sequence of an MHC class I epitope, or a portion thereof.

In some embodiments, a synthetic polypeptide comprises a first peptide where the first peptide comprises an amino acid sequence of an HLA-A epitope. In certain embodiments, the first peptide comprises the amino acid sequence X₁X₂X₃X₄X₅X₆X₇X₈X₉, wherein X₁ is F, Y, G, I, L, K, M or V; X₂ is L, M or V; X₃ is L, S, K, A, Y, F, P, M, or R; X₄ is E, P, K, G, D or T; X₅ is I, L, E, G, K, Y, N, F, V or H; X₆ is E, A, L, I, V or T; X₇ is P, S, A, Y or H; X₈ is E, P, A, K, or S; and X₉ is W, L, or V; using standard single letter amino acid abbreviations. In certain embodiments, the first peptide comprises an amino acid sequence selected from FVLELEPEWT, FVLELEPEWTV, YLSPIASPL, YLSPIASPLL, GLLPELPAV, GLKEGIPAL and AAFIFILTV.

In some embodiments, a synthetic polypeptide comprises a first peptide where the first peptide comprises an amino acid sequence of an HLA-B epitope. In certain embodiments, the first peptide comprises the amino acid sequence LSSPVTKSF.

In some embodiments, a synthetic polypeptide comprises a first peptide where the first peptide comprises an amino acid sequence of an HLA-C epitope. In certain embodiments, the first peptide comprises the amino acid sequence GAVDPLLAL.

In some embodiments, a synthetic polypeptide comprises a first peptide where the first peptide comprises an amino acid sequence comprising amino acids that are conserved among HLA-A, HLA-B and HLA-C epitopes. For example, in certain embodiments, a first peptide comprises a consensus amino acid sequence derived from aligning the amino acid sequences of an HLA-A epitope, HLA-B epitope and an HLA-C epitope. In certain embodiments, a first peptide comprises a consensus amino acid sequence derived from aligning multiple amino acid sequences derived from HLA-A epitopes, HLA-B epitopes and an HLA-C epitopes. In some embodiments, the amino acid sequence of a first peptide comprises amino acids that are highly conserved among HLA-A epitopes, HLA-B epitopes and an HLA-C epitopes.

In some embodiments, a synthetic polypeptide comprises a first peptide where the first peptide comprises an amino acid sequence of a non-classical HLA epitope. In some embodiments, a synthetic polypeptide comprises a first peptide where the first peptide comprises an amino acid sequence of an HLA-E or HLA-G epitope.

In some embodiments, a synthetic polypeptide comprises one additional amino acid. In some embodiments, a synthetic polypeptide comprises no more than one additional amino acid where the additional amino acid is covalently linked to the C-terminus of the first peptide. In certain embodiments, the additional amino acid comprises an electronegative or electropositive charged side group. In some embodiments, an electronegative charged side group can be a carboxyl group. In some embodiments, an electropositive charged side group can be a primary, secondary or tertiary amine. In certain embodiments, the additional amino acid is aspartic acid, glutamate, or a variant or derivative thereof. In certain embodiments, the additional amino acid is lysine, or histidine.

In some embodiments, a synthetic polypeptide comprises a second peptide. In certain embodiments, a second peptide comprises or consists of 2 to 7 amino acids. A second peptide may include 2, 3, 4, 5, 6, or 7 amino acids and may be 2, 3, 4, 5, 6, or 7 amino acids in length. In some embodiments, a second peptide is 2 amino acids in length. In some embodiments, a second peptide is 3 amino acids in length. In some embodiments, a second peptide is not more than 3 amino acids in length. A second peptide is often covalently linked to the C-terminus of the first peptide of a synthetic polypeptide. In some embodiments, the second peptide is covalently linked to the first peptide by a peptide bond. In certain embodiments, the second peptide comprises at least one amino acid having an electronegative or electropositive charged side group. In some embodiments, an electronegative charged side group can be a carboxyl group. In some embodiments, an electropositive charged side group can be a primary, secondary or tertiary amine. In some embodiments, the second peptide comprises one or more amino acids selected from D, E, K or H. In certain embodiments, the amino acid sequence of the second peptide is selected from the group consisting of D, K, DN, DG, GGNE, DGK, and DGKSLR.

In certain embodiments, the second peptide sequence comprises glycine or serine. In some embodiments, the second peptide comprises valine, alanine or leucine.

A synthetic polypeptide is typically 8 to 18 amino acids in length. In some embodiments, a synthetic polypeptide consists of 8 to 18 amino acids in length. In some embodiments, a synthetic polypeptide is at least 8 amino acids in length and not more than 10, not more than 11, not more than 12, not more than 13, or not more than 14 amino acids in length. In certain embodiments, a synthetic polypeptide comprises or consists of an amino acid sequence selected from the group consisting of AAGIGILTVK, AAGIGILTVD, AAGIGILTVDGK, LSSPVTKSFK, LSSPVTKSFD, LSSPVTKSFDGK, GAVDPLLALK, GAVDPLLALD, and GAVDPLLALDGK.

In some embodiments, a synthetic polypeptide binds specifically to an MHC class I molecule. In some embodiments, a synthetic polypeptide upon binding to an MHC class I molecule, induces a structural change in the MHC class I molecule, and the structural change comprises opening an F pocket of the MHC class I molecule. An F pocket is shown and described in the Examples and Figures herein. An F pocket is a terminal portion of the binding groove in an MHC class I molecule that can accept and bind to the C-terminal portion of an MHC class I epitope (MHC class I binding peptide). As described herein, extending the amino acid of an MHC class I epitope by the addition of at least one charged amino acid induces structural changes in the F pocket of the MHC class I molecule. The structural changes observed include an open confirmation of the F pocket that allows the C-terminal charged amino acids of a bound synthetic polypeptide to extend away from the F pocket and into the solvent.

In some embodiments, binding of a synthetic polypeptide to an MHC class I molecule blocks, abrogates, reduces, decreases or inhibits binding of a killer immunoglobulin receptor (KIR) to the MHC class I molecule. A killer immunoglobulin receptor (KIR) is sometimes an inhibitor KIR. KIR are typically expressed on an NK cell.

A synthetic polypeptide can be generated, manufactured or produced by a suitable method. For example, a synthetic polypeptide can be made using any suitable peptide liquid phase or solid phase peptide synthesis procedure. In certain embodiments, a synthetic polypeptide is not found in nature and is not naturally occurring. In some embodiments, a naturally occurring protein does not comprise a synthetic polypeptide described herein. However, a synthetic polypeptide described herein may be part of a larger protein, or may be present as an isolated synthetic peptide.

In certain embodiments, an amino acid sequence of a synthetic polypeptide is designed or modified to optimize binding affinity for a target (e.g., MHC Class I), solubility and/or function. Synthetic polypeptides can be generated and screened for binding to MHC Class I molecules. Certain screen methods may include determining a structure of an MHC Class I molecule bound to a synthetic polypeptide. In some embodiments a structure is determined by a suitable crystallographic method.

In certain embodiments a synthetic polypeptide is modified to include certain amino acid additions, substitutions, or deletions designed or intended, for example, to reduce susceptibility of a synthetic polypeptide to proteolysis, reduce susceptibility of a synthetic polypeptide to oxidation, increase serum half-life and/or confer or modify other physicochemical, pharmacokinetic or functional properties of a synthetic polypeptide.

The term “specifically binds” refers to a synthetic polypeptide that binds a Class I MHC molecule in preference to binding other molecules or other peptides as determined by, for example, a suitable in vitro assay (e.g., an Elisa, Immunoblot, Flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

In some embodiments a synthetic polypeptide that specifically binds to an MHC Class I molecule, or a portion thereof, is a synthetic polypeptide that binds with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In some embodiments a synthetic polypeptide that specifically binds to an MHC Class I molecule, or a portion thereof, is a synthetic polypeptide that binds an MHC Class I molecule, or a portion thereof, with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM.

In certain embodiments, a macromolecule comprises a synthetic polypeptide. For example, in certain embodiment, a synthetic polypeptide is part of a larger molecule or is attached, linked or covalently bound to one or more compounds or molecules to form a macromolecule. A macromolecule may comprise at least one synthetic polypeptide and one or more molecules selected from antibodies, proteins (e.g., carrier protein, transport proteins, antigens), adjuvants (e.g., endotoxin, bacterial cell wall proteins), toxins, sugars, starches, fatty acids, scaffolds, structural polypeptides, therapeutic agents, labels, linkers, the like or combinations thereof. A macromolecule may comprise one or more synthetic polypeptides and/or one or more proteins. In some embodiments a macromolecule comprises 2, 3, 4 or more synthetic polypeptides. In some embodiments a macromolecule comprises two synthetic polypeptides. In some embodiments a macromolecule comprises one or more structural portions (e.g., scaffolds, structural polypeptides, linkers). A macromolecule may comprise any suitable scaffold, non-limiting examples of which include scaffolds derived from an antibody, a Z domain of Protein A, gamma-B crystalline, ubiquitin, cystatin, Sac7d, a triple helix coiled coil, a lipocalin, an ankyrin repeat motif, an SH3 domain of Fyn, a Kunitz domain of a suitable protease inhibitor, a fibronectin domain, a nucleic acid polymer, the like, portions thereof or combinations thereof. In some embodiments a macromolecule does not comprise a scaffold. In some embodiments macromolecule comprises a substrate and a synthetic polypeptide. In certain embodiments, a synthetic polypeptide is attached to a substrate (e.g., a polymer, a non-organic material, silicon, a bead, or the like).

In some embodiments a synthetic polypeptide comprises a label. As used herein, the terms “label” or “labeled” refers to incorporation of a detectable marker, e.g., by incorporation of a labeled amino acid or attachment to a polypeptide of biotin moieties that can be detected by labeled avidin (e.g., streptavidin containing a fluorescent marker or enzymatic activity that can be detected by optical or colorimetric methods). In certain embodiments, a label or marker can be attached to a synthetic polypeptide to generate a therapeutic or diagnostic agent. A synthetic polypeptide can be attached covalently or non-covalently to any suitable label or marker. Various methods of labeling polypeptides and glycoproteins are known to those skilled in the art and can be used. Non-limiting examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹²⁵I, ¹³¹I), fluorescent labels, enzymatic labels (e.g., horseradish peroxidase, β-galactosidase, luciferase, alkaline phosphatase), chemiluminescent labels, a metallic label, a chromophore, an electro-chemiluminescent label, a phosphorescent label, a quencher (e.g., a fluorophore quencher), a fluorescence resonance energy transfer (FRET) pair (e.g., donor and acceptor), a dye, an enzyme substrate, a small molecule, a mass tag, quantum dots, nanoparticles, biotinyl groups, predetermined polypeptide epitopes recognized by a secondary reporter (e.g., leucine zipper pair sequences, binding sites for secondary antibodies, metal binding domains, epitope tags), the like or combinations thereof.

In some embodiments a synthetic polypeptide comprises a suitable therapeutic agent. A synthetic polypeptide can be attached covalently or non-covalently to any suitable therapeutic agent. Non-limiting examples of a therapeutic agent include a medication, toxin, radioisotope, ligand, receptor, cytokine, antibody, anti-neoplastic agent, inhibitor (e.g., a receptor antagonist, an enzyme inhibitor), the like or combinations thereof. Accordingly, in certain embodiments, a synthetic polypeptide disclosed herein comprises an anti-neoplastic agent.

In some embodiments a synthetic polypeptide comprises a suitable carrier. A synthetic polypeptide can be attached covalently or non-covalently to a suitable carrier. Non-limiting examples of a carrier include agents or molecules that alter or extend the in vivo half-life of a synthetic polypeptide include polyethylene glycol, glycogen (e.g., by glycosylation of a synthetic polypeptide), a dextran, a carrier or vehicle described in U.S. Pat. No. 6,660,843, the like or combinations thereof.

In some embodiments a label, therapeutic agent or carrier is bound to a synthetic polypeptide by use of a suitable linker. Non-limiting examples of a suitable linker include silanes, thiols, phosphonic acid, polyethylene glycol (PEG), amino acids and peptides, polymers thereof, derivatives thereof, the like and combinations thereof. Methods of attaching two or more molecules using a linker are to those skilled in the art and are sometimes referred to as “crosslinking.”

In some embodiments a label, therapeutic agent, carrier or linker is attached to a suitable thiol group of a synthetic polypeptide (e.g., a thiol group of a cysteine residue). Other non-limiting examples of attaching a label, therapeutic agent, carrier and/or linker to a synthetic polypeptide include reacting an amine with an N-hydroxysuccinimide (NHS) ester, an imidoester, a pentafluorophenyl (PFP) ester, a hydroxymethyl phosphine, an oxirane or any other carbonyl compound; reacting a carboxyl with a carbodiimide; reacting a sulfhydryl with a maleimide, a haloacetyl, a pyridyldisulfide, and/or a vinyl sulfone; reacting an aldehyde with a hydrazine; reacting any non-selective group with diazirine and/or aryl azide; reacting a hydroxyl with isocyanate; reacting a hydroxylamine with a carbonyl compound; the like and combinations thereof.

In some embodiments, presented herein is a method of modulating activity of a Natural Killer cell (NK cell) by contacting the NK cell with a synthetic polypeptide, macromolecule, compound or composition provided herein. In certain embodiments, the method comprises activating NK cell activity. In particular embodiments, the NK cell is in a subject, including but not limited to a subject having or suspected of having, a neoplasia, neoplastic disorder, tumor, cancer, or malignancy or having or suspected of having an infection or an infection disease.

In some embodiments, presented herein is a method of treating a subject having or suspected of having, a neoplasia, neoplastic disorder, tumor, cancer, or malignancy. In certain embodiments, a method of treating a subject comprises administering a therapeutically effective amount of a synthetic polypeptide to a subject. In certain embodiments, a method of treating a subject comprises administering a therapeutically effective amount of a macromolecule to a subject. In certain embodiments, a method comprises reducing or inhibiting proliferation of a neoplastic cell, tumor, cancer or malignant cell, comprising contacting the cell, tumor, cancer or malignant cell, with the synthetic polypeptide in an amount sufficient to reduce or inhibit proliferation of the neoplastic cell, tumor, cancer or malignant cell.

In some embodiments, a method of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprises administering to a subject an amount of a synthetic polypeptide sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

Non-limiting examples of a neoplasia, neoplastic disorder, tumor, cancer or malignancy include a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma. A neoplasia, neoplastic disorder, tumor, cancer or malignancy may comprise or involve hematopoietic cells. Non-limiting examples of a sarcoma include a lymphosarcoma, liposarcoma, osteosarcoma, chondrosarcoma, leiomyosarcoma, rhabdomyosarcoma or fibrosarcoma. In some embodiments, a neoplasia, neoplastic disorder, tumor, cancer or malignancy is a myeloma, lymphoma or leukemia. In some embodiments, a neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer. In some embodiments, a neoplasia, neoplastic disorder, tumor, cancer or malignancy comprises a small cell lung or non-small cell lung cancer. In some embodiments, a neoplasia, neoplastic disorder, tumor, cancer or malignancy comprises a stem cell neoplasia, tumor, cancer or malignancy. In some embodiments, a neoplasia, neoplastic disorder, tumor, cancer or malignancy.

In some embodiments, a method inhibits, or reduces relapse or progression of the neoplasia, neoplastic disorder, tumor, cancer or malignancy. In some embodiments, a method comprises administering an anti-cell proliferative, anti-neoplastic, anti-tumor, anti-cancer or immune-enhancing treatment or therapy. In some embodiments, a method of treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan. In some embodiments, a method of treatment results in reducing or decreasing severity, duration or frequency of an adverse symptom or complication associated with or caused by the neoplasia, tumor, cancer or malignancy. In some embodiments, a method of treatment results in reducing or decreasing pain, discomfort, nausea, weakness or lethargy. In some embodiments, a method of treatment results in increased energy, appetite, improved mobility or psychological well being.

In certain embodiments, a method herein comprises contacting a cell of a subject with a synthetic polypeptide disclosed herein.

In certain embodiments, a pharmaceutical composition comprises a synthetic polypeptide or compound described herein. A pharmaceutical composition can be formulated for a suitable route of administration. In some embodiments a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants (Remington's Pharmaceutical Sciences, 18th Ed., A. R. Gennaro, ed., Mack Publishing Company (1995)).

In certain embodiments, a pharmaceutical composition comprises a suitable excipient, non-limiting example of which include anti-adherents (e.g., magnesium stearate), a binder, fillers, monosaccharides, disaccharides, other carbohydrates (e.g., glucose, mannose or dextrins), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington's Pharmaceutical Sciences, 18th Ed., A. R. Gennaro, ed., Mack Publishing Company (1995). The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art. In some embodiments, a pharmaceutical composition comprises a binder.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent includes those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition, pharmaceutical composition or synthetic polypeptide is substantially free of blood, or a blood product contaminant (e.g., blood cells, platelets, polypeptides, minerals, blood borne compounds or chemicals, and the like). In some embodiments a composition, pharmaceutical composition or synthetic polypeptide is substantially free of serum and serum contaminants (e.g., serum proteins, serum lipids, serum carbohydrates, serum antigens and the like). In some embodiments a composition, pharmaceutical composition or synthetic polypeptide is substantially free a pathogen (e.g., a virus, parasite or bacteria). In some embodiments a composition, pharmaceutical composition or synthetic polypeptide is substantially free of endotoxin. In some embodiments a composition, pharmaceutical composition or synthetic polypeptide is sterile. In certain embodiments, a composition or pharmaceutical composition comprises a synthetic polypeptide and a diluent (e.g., phosphate buffered saline). In certain embodiments, a composition or pharmaceutical composition comprises a synthetic polypeptide and an excipient, (e.g., sodium citrate dehydrate, or polyoxyethylene-sorbitan-20 mono-oleate (polysorbate 80)).

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parental administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), and the like). In certain embodiments, reconstituted forms of a lyophilized pharmaceutical composition are suitable for parental administration (e.g., intravenous administration) to a mammal.

In some embodiments a pharmaceutical compositions described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a synthetic polypeptide from a topical patch.

In certain embodiments, an optimal pharmaceutical composition will be determined by one skilled in the art depending upon, for example, the intended route of administration, delivery format and desired dosage (see e.g., Remington's Pharmaceutical Sciences, supra). In certain embodiments, such compositions may influence the physical state, stability, rate of in vivo release and rate of in vivo clearance of the antibodies of the invention.

In some embodiments a composition, pharmaceutical composition or synthetic polypeptide described herein is used to treat a subject having or suspected of having a neoplastic disorder or cancer. In certain embodiments, a synthetic polypeptide or pharmaceutical composition described herein is used in treating a neoplastic disorder or cancer in a subject. In some embodiments, presented herein is a method of treating a subject having or suspected of having a neoplastic disorder or cancer. In certain embodiments, a method of treating a subject having or suspected of having a neoplastic disorder or cancer comprises administering a therapeutically effective amount of a composition, pharmaceutical composition or synthetic polypeptide described herein to the subject. In certain embodiments, a method of treatment comprises contacting a cell (e.g., one or more cells) of a subject with a therapeutically effective amount of a composition, pharmaceutical composition or synthetic polypeptide described herein. A cell of a subject may be found inside a subject (e.g., in vivo) or outside the subject (e.g., in vitro or ex vivo).

A composition, pharmaceutical composition or synthetic polypeptide disclosed herein can be used to treat a suitable neoplastic order or cancer involving a cell type that expresses an MHC Class I molecule. Non-limiting examples of a neoplastic disorder or cancer that can be treated by a method herein includes a lung carcinoma, breast carcinoma, ovarian carcinoma, kidney carcinoma, colorectal carcinoma, gastric carcinoma, thyroid carcinoma, pancreas carcinoma, neuroblastoma, or a squamous cell carcinoma of the head and neck, cervical cancer, hepatocellular cancer, sarcomas, mesothelioma, glioblastoma, multiple myeloma, melanoma, prostate and esophageal carcinoma. Non-limiting examples of lymphomas include B-cell lymphomas, (e.g., diffuse large B-cell lymphoma, primary mediastinal B-cell lymphoma, intravascular large B-cell lymphoma, and the like), follicular lymphoma, chronic lymphocytic leukemia/small lymphocytic lymphoma, mantle cell lymphoma, marginal zone B-cell lymphomas (e.g., extranodal marginal zone B-cell lymphomas, also known as mucosa-associated lymphoid tissue (malt) lymphomas, nodal marginal zone B-cell lymphoma, splenic marginal zone B-cell lymphoma and the like), Burkitt lymphoma, Hodgkins lymphoma, lymphoplasmacytic lymphoma (waldenstrom macroglobulinemia), hairy cell leukemia, primary central nervous system (cns) lymphoma, T-cell lymphomas, (e.g., non-hodgkin lymphomas, precursor T-lymphoblastic lymphoma/leukemia, peripheral T-cell lymphomas, (e.g., cutaneous t-cell lymphomas, adult T-cell leukemia/lymphoma, angioimmunoblastic T-cell lymphoma, extranodal natural killer/T-cell lymphoma, enteropathy-associated intestinal t-cell lymphoma, anaplastic large cell lymphoma, and the like).

Any suitable method of administering a composition, pharmaceutical composition or synthetic polypeptide to a subject can be used. The exact formulation and route of administration for a composition for use according to the methods of the invention described herein can be chosen by the individual physician in view of the patient's condition. See, e.g., Fingl et al. 1975, in “The Pharmacological Basis of Therapeutics,” Ch. 1, p. 1; which is incorporated herein by reference in its entirety. Any suitable route of administration can be used for administration of a pharmaceutical composition or a synthetic polypeptide described herein. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is sometimes provided to a subject for self-administration or for administration to a subject by another (e.g., a non-medical professional). For example a composition described herein can be provided as an instruction written by a medical practitioner that authorizes a patient to be provided a composition or treatment described herein (e.g., a prescription). In another example, a composition can be provided to a subject where the subject self-administers a composition orally, intravenously or by way of an inhaler, for example.

Alternately, one can administer compositions for use according to the methods of the invention in a local rather than systemic manner, for example, via direct application to the skin, mucous membrane or region of interest for treating, including using a depot or sustained release formulation.

In some embodiments a pharmaceutical composition comprising a synthetic polypeptide can be administered alone (e.g., as a single active ingredient (AI or e.g., as a single active pharmaceutical ingredient (API)). In other embodiments, a pharmaceutical composition comprising a synthetic polypeptide can be administered in combination with one or more additional AIs/APIs, for example, as two separate compositions or as a single composition where the one or more additional AIs/APIs are mixed or formulated together with the synthetic polypeptide in a pharmaceutical composition.

A pharmaceutical composition can be manufactured by any suitable manner, including, e.g., by means of conventional mixing, dissolving, granulating, dragee-making, levigating, emulsifying, encapsulating, entrapping or tableting processes.

In some embodiments a pharmaceutical composition comprising a synthetic polypeptide is administered at a suitable frequency or interval as needed to obtain an effective therapeutic outcome. An effective therapeutic outcome can be determined by monitoring the number, viability, growth, mitosis, or metastasis of neoplastic or cancerous cells in a subject affected with a neoplastic disorder or cancer. Accordingly, in certain embodiments, a decrease in the number, viability, growth, mitosis, or metastasis of neoplastic or cancerous cells in a subject is considered an effective therapeutic outcome. In some embodiments, a pharmaceutical composition comprising a synthetic polypeptide can be administered hourly, once a day, twice a day, three times a day, four times a day, five times a day, and/or at regular intervals, for example, every day, every other day, three times a week, weekly, every other week, once a month and/or simply at a frequency or interval as needed or recommended by a medical professional.

In some embodiments, an amount of a synthetic polypeptide in a composition is an amount needed to obtain an effective therapeutic outcome. In certain embodiments, the amount of a synthetic polypeptide in a composition (e.g., a pharmaceutical composition) is an amount sufficient to prevent, treat, reduce the severity of, delay the onset of, and/or alleviate a symptom of a neoplastic disorder or cancer, as contemplated herein.

A “therapeutically effective amount” means an amount sufficient to obtain an effective therapeutic outcome and/or an amount necessary sufficient to prevent, treat, reduce the severity of, delay the onset of, and/or alleviate a symptom of a neoplastic disorder or cancer. In certain embodiments, a “therapeutically effective amount” means an amount sufficient to terminate the grow of, and/or slow the growth of a neoplasm or cancer. In certain embodiments, a “therapeutically effective amount” means an amount sufficient to inhibit the replication of, and/or induce the death of one or more neoplastic cells. Determination of a therapeutically effective amount is well within the capability of those skilled in the art, especially in light of the detailed disclosure provided herein.

In some embodiments, an amount of a synthetic polypeptide in a composition is an amount that is at least a therapeutically effective amount and an amount low enough to minimize unwanted adverse reactions. The exact amount of a synthetic polypeptide or combinations of active agents required will vary from subject to subject, depending on age, weight, and general condition of a subject, the severity of the condition being treated, and the particular combination of drugs administered. Thus, it is not always possible to specify an exact therapeutically effective amount to treat a neoplastic disorder in a diverse group of subjects. As is well known, the specific dosage for a given patient under specific conditions and for a specific disease will routinely vary, but determination of the optimum amount in each case can readily be accomplished by simple routine procedures. Thus, a therapeutically effective amount of a synthetic polypeptide used to treat a neoplastic disorder may be determined by one of ordinary skill in the art using routine experimentation.

In certain embodiments, an amount of a synthetic polypeptide in a composition is administered at a suitable therapeutically effective amount or a dose (e.g., at a suitable volume and concentration, which sometimes depends, in part, on a particular route of administration). Within certain embodiments, a synthetic polypeptide (e.g., a synthetic polypeptide in a composition) can be administered at a dose from about 0.0001 mg/kg (e.g., per kg body weight of a subject) to 500 mg/kg, 0.001 mg/kg to 500 mg/kg, 0.001 mg/kg to 500 mg/kg, 0.01 mg/kg to 500 mg/kg, 0.1 mg/kg to 500 mg/kg, 0.1 mg/kg to 400 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of a synthetic polypeptide can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments a therapeutically effective amount of a synthetic polypeptide is between about 0.1 mg/kg to 500 mg/kg, or between about 1 mg/kg and about 300 mg/kg. Volumes suitable for intravenous administration are well known.

Any suitable method can be used to detect and/or quantitate the presence, absence and/or amount of a synthetic polypeptide specifically bound to cMET, or a portion thereof, non-limiting examples of such methods can be found in Immunology, Werner Luttmann; Academic Press, 2006 and/or Medical Detection and Quantification of Antibodies to Biopharmaceuticals: Practical and Applied Considerations, Michael G. Tovey; John Wiley & Sons, Jul. 12, 2011. Additional non-limiting examples of methods that can be used to detect and/or quantitate the presence, absence and/or amount of a synthetic polypeptide specifically bound to cMET, or a portion thereof, include use of a competitive immunoassay, a non-competitive immuno assay, western blots, a radioimmunoassay, an ELISA (enzyme linked immunosorbent assay), a competition or sandwich ELISA, a sandwich immunoassay, an immunoprecipitation assay, an immunoradiometric assay, a fluorescent immunoassay, a protein A immunoassay, a precipitin reaction, a gel diffusion precipitin reaction, an immunodiffusion assay, an agglutination assay, a complement fixation assay, an immunohistochemical assay, a Western blot assay, an immunohistological assay, an immunocytochemical assay, a dot blot assay, a fluorescence polarization assay, a scintillation proximity assay, a homogeneous time resolved fluorescence assay, an IAsys analysis, a BIAcore analysis, the like or a combination thereof.

A pharmaceutical composition comprising an amount or dose of a synthetic polypeptide can, if desired, be provided in a kit, pack or dispensing device, which can contain one or more doses of a synthetic polypeptide. The pack can for example comprise metal or plastic foil, such as a blister pack. The pack or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments a kit or pack comprises an amount of a synthetic polypeptide sufficient to treat a patient for 1 day to 1 year, 1 day to 180 days, 1 day to 120 days, 1 day to 90 days, 1 day to 60 days, 1 day to 30 days, or any day or number of days there between, 1-4 hours, 1-12 hours, or 1-24 hours.

A kit optionally includes a product label or packaging inserts including a description of the components or instructions for use in vitro, in vivo, or ex vivo, of the components therein. Exemplary instructions include instructions for a diagnostic method, treatment protocol or therapeutic regimen. In certain embodiments, a kit comprises packaging material, which refers to a physical structure housing components of the kit. The packaging material can maintain the components sterilely, and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein. Product labels or inserts can include information on potential adverse side effects and/or warnings.

In some embodiments, a kit is a diagnostic kit comprising a synthetic polypeptide. A synthetic polypeptide comprised in a diagnostic kit can take any suitable form. In some embodiments, a diagnostic comprises a synthetic polypeptide and a detectable label. In certain embodiments, for example, a diagnostic kit comprises or consists of a stick test, including necessary reagents to perform the method of the invention and to produce, for example, a colorimetric result which can be compared against a color chart or standard curve.

### EXAMPLES

### Example 1

### Introduction

Toxoplasmosis is a parasitic disease caused by infection with the large intracellular protozoan Toxoplasma gondii. While generally asymptomatic in healthy adults, T. gondii infection can cause congenital toxoplasmosis during pregnancy and result in abortion or neonatal diseases. T cell mediated immunity against T. gondii derived peptide antigens provides strong protection against T. gondii and involves both peptide presentation by Major Histocompatibility Class I (MHC I) and Class II (MHC II) proteins. While T. gondii can interfere with CD4 T cell responses by downregulating MHC II expression in IFN-γ activated macrophages, immunization with T. gondii MHC II peptide ligands can elicit potent CD4 T cell response that can lower parasite burden in the brain. Immunocompromised individuals and patients with T-cell deficiencies are highly susceptible to T. gondii infections.

CD8 T cell responses have been studies more widely than CD4 and peptide ligands for MHC I have been identified to be derived from surface proteins, or proteins of specialized secretory organelles (rhoptry proteins) that can either be secreted into the parasite cytosol or the parasitophorous vacuole.

HLA-A*02:01 has been the focus of studies aimed at identifying peptide ligands that confer protection against T. gondii in HLA-A*02:01 transgenic mice. T. gondii peptides, like most canonical peptide ligands for MHC I are 9-10 amino acids in length. These long peptides interact with the residues of the binding grove of HLA class I heavy (alpha) chain much like the canonical binders with some changes. The second (P2) and C-terminal (PS) residues of the antigen peptide anchor into the A and F pockets of the binding grove, respectively while the middle portion of these over-sized peptides either ‘bulge out’ or ‘zig-zag’ in the binding groove to be accommodated.

In contrast to these ‘bulged’ peptides, longer T. gondii peptides were identified that elute from HLA-A*02:01 molecules and have a conserved N-terminal start but differed in length at the C-terminus. The inventors have shown through crystallographic studies that in the HLA-A*02:01 complex with one 12 mer peptide residue Tyr84 of the heavy chain swung out and opened the F pocket, allowing the C-terminal amino acid to protrude into the solvent, while the corresponding 11 mer peptide bound in a conventional zig-zag orientation tucked with both peptide ends inside the peptide binding groove.

To further investigate whether the opening of the binding grove could be achieved with other peptides identified herein, the inventors crystallized complexes of HLA-A*02:01 with several pairs of core (nested) and C-terminally extended peptides. The present invention provides that there are at least two distinct modes of opening the F pocket of HLA-A*02:01 involving the residues Tyr84 and Lys146.

Since peptide presentation by MHC controls immune responses against infection, cancer and also autoimmunity, an understanding the mechanism of antigen presentation by MHC I allowed the inventors to design therapeutic strategies aimed at modulating subsequent immune responses to control disease, disclosed therein.

### Methods:

### HLA-A*02:01 Expression and Purification for Crystallization

HLA-A*02:01 class I heavy chain ectodomain (residues 21-274) and human β-2 microglobulin (hβ2m, 1-99) were expressed as inclusions bodies and refolded with modifications reported herein. Briefly, both the heavy chain and light chain were expressed in Escherichia coli BL21 DE3 cells, induced at OD600 of 0.6 with 1 mM isopropyl-1-thio-D-galactopyranoside (IPTG) and cells were harvested after 4 hours by centrifugation (5000 g for 20 mins). Cells were resuspended separately in lysis buffer (100 mM Tris-HCl pH 7.0, 5 mM EDTA, 5 mM DTT, 0.5 mM PMSF) and the cells were broken the cells were broken with 4-5 passes through a microfluidizer (20 kPa) (Microfluidics). Cell lysate was centrifuged (50,000 g for 30 min at 4° C.) to collect inclusion bodies. Inclusion bodies were further resuspended in wash buffer A (100 mM Tris-HCl pH 7.0, 5 mM EDTA, 5 mM DTT, 2 M Urea, 2% w/v Triton X-100) centrifuged again and washed in wash buffer B (100 mM Tris-HCl pH 7.0, 5 mM EDTA, 2 mM DTT). Finally the inclusion bodies were denatured in extraction buffer (50 mM Tris-HCl pH 7.0, 5 mM EDTA, 2 mM DTT, 6 M Guanidine-HCl) for subsequent refolding. 3 mg of hβ2m was added dropwise to 250 mL of refolding buffer (0.1 M Tris-HCl pH 8.0, 2 mM EDTA, 400 mM L-arginine, 5 mM oxidized glutathione, 5 mM reduced glutathione) and stirred for 1-2 hours. Between 11-15 mg of HLA-A heavy chain mixed with 2-3 mg of individual peptide (GenScript) was then added to the refolding mix and further stirred at 4° C. for 72 hours. Final heavy chain:light chain:peptide ratios were in the range of 2:1:12 and 2.5:1:12 for different peptides. Following refolding, the refolding mixture was centrifuged at 50,000 g to remove any precipitated protein and the supernatant was concentrated to about 3 ml for size exclusion chromatography (SEC) using a Superdex S200 HR16/60 gel filtration column pre-equilibrated with SEC buffer (20 mM Tris-HCl pH 7.5, 150 mM NaCl). Fractions containing refolded HLA-A*02:01-β2m-peptide complexes were pooled, concentrated to 5-12 mg/mL and used for subsequent crystallization experiments.

### Crystallization and Data Collection

Initial attempts were performed to obtain crystals for the HLA-A*02:01-peptide complexes using factorial screens. Thin needle shaped sea urchin crystals of HLA-A*02:01-F12K complex obtained in 1.2M sodium citrate were used to cross seed the other complexes. The complexes were equilibrated in 30% PEG 4000, 0.1M Tris-HCl pH 8.0, 0.2M lithium sulfate for 1-2 hours by mixing 0.15 μl complex and 0.15 μl of precipitant at 20° C. before seeding. Thin plate-like crystals were obtained by sitting drop vapor diffusion at 20° C. after 2-4 days. The crystals were flash frozen in cryoprotectant (crystallization solution: 100% glycerol—3:1) using liquid nitrogen.

Diffraction data for HLA-A*02:01 complex with peptides G9V, G11N, G13E and Y16R were collected remotely at beamline 7.1 at the Stanford Synchrotron Radiation Light source (SSRL) and processed to 1.86 Å, 2.3 Å, 2.1 Å and 2.4 Å resolution, respectively using HKL2000. Diffraction data for HLA-A*02:01 complex with peptides G9L, Y9L and Y10L were collected remotely at beamline 12.3.1 at the Advanced Light Source (ALS) and processed to 1.85 Å, 2.5 Å and 2.75 Å resolution, respectively using HKL2000. Phases were obtained by molecular replacement with Phaser MR in ccp4i using the protein coordinates for HLA-A*02:01 (PDB ID 3MRE) and resulted in unambiguous electron density for all the peptides. Model building was carried out using COOT. Structures were refined using Refmac. Data collection and structure refinement parameters are provided in Table 1.

### Thermal Denaturation Assay

HLA-A*02:01-β2m-peptide complexes with the various different peptides were analyzed for thermal denaturation by differential scanning fluorimetry using a LightCycler 480 (Roche). HLA-A*02:01-β2m-peptide complexes with different peptides at 100 μM in reaction buffer (20 mM Tris-HCl pH 7.5, 150 mM NaCl) were used as stock solutions. Each reaction mixture constituted of 1-2 μl protein complex stock solution, 2l of SYPRO Orange dye (100×, Invitrogen) made up to 20 μl in reaction buffer in a 96-well white plate compatible with the instrument. A temperature gradient from 20° C.-85° C. at steps of 0.06° C./sec and 10 acquisitions/° C. was used for the experiment. Each experiment with individual protein-peptide complex was repeated thrice. A melt curve of the total fluorescence of the run was plotted against temperature. The minima of the first derivative of the melt curve from raw fluorescence data (temperature differential of absolute fluorescence versus temperature) provided the meting temperature (Tm) for individual HLA-A*02:01-β2m peptide complexes (inflection point of the melt curve).

### Results:

In contrast to previous studies, in which the extending peptide (FVLELEPEWTVK) had a single lysine added to the C-terminus of the core peptide (FVLELEPEWTV) and induced a structural change in Tyr84 of HLA-A*02:01, most other peptides had C-terminal amino acid additions that contained negatively charged amino acids or both negatively and positively charged residues.

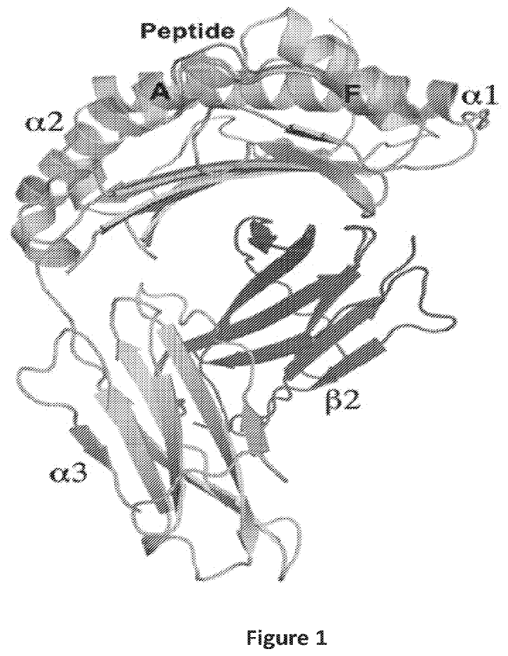

To investigate if the F pocket of HLA-A*02:01 could also be opened by these extending peptides, the inventors refolded HLA-A*02:01 with several nested and extending peptides and determined the crystal structures of these complexes. The inventors obtained crystal structures for all complexes at resolutions between 1.85 Å and 2.75 Å (Table 1). Electron densities for all the peptides were well defined over the entire peptide length that is bound within the binding groove, while C-terminally extending residues that did not contact HLA-A*02:01 were disordered (FIG. 1). When all the different peptides are compared, slight structural changes in HLA-A*02:01 are observed in the A pocket. Peptides with an N-terminal tyrosine (YLSPIASPL, YLSPIASPLL, and YLSPIASPLLDGKSLR) open the A pocket slightly for the bulky side chain to be accommodated, while peptides that begin with glycine (GLKEGIPAL, GLKEGIPALDN, GLLPELPAV, and GLLPELPAVGGNE) are more buried inside the A pocket, since they lack any side chain (FIG. 1). In addition, subtle structural changes are observed throughout the binding groove to allow optimal binding of the different amino acid side chains. However, when structures of the core peptides are compared with their respective extended peptides, the position of Tyr84 of HLA-A*02:01 was unchanged. Surprisingly, however, Lys146 of the F pocket, which is located close to Tyr84 and forms a “lid” to bury the P9 amino acid in the core peptides moved upwards to open the F pocket, when the extending peptides were bound (FIG. 1). While Lys146 adopts slightly different positions when all the extending peptide structures are compared, in each structure the Lys146 lid was opened for the C-terminal extensions to protrude from the F pocket.

### Hydrogen Bond Network for Nested and Longer Peptide Pairs

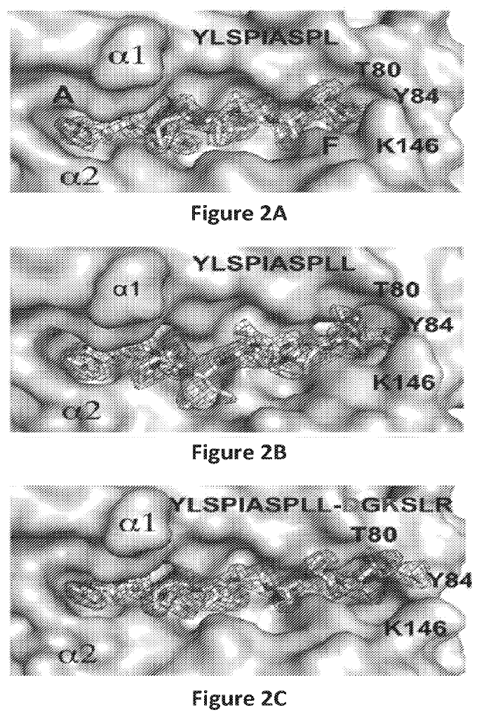

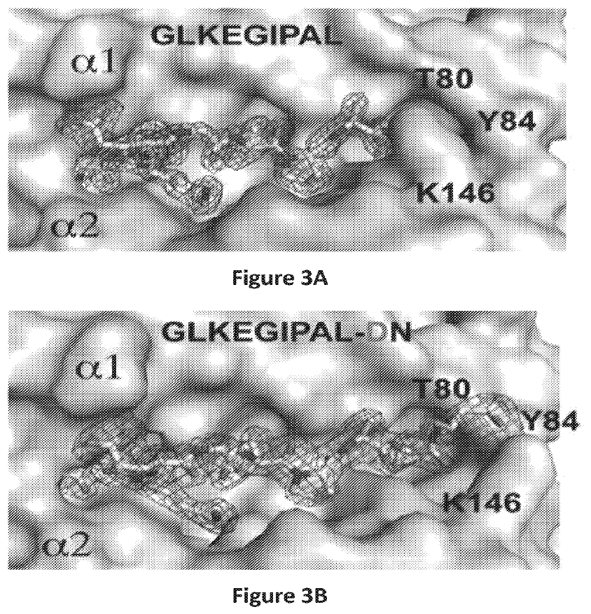

Next, the inventors looked at the detailed interactions between HLA-A*02:01 and the individual peptides. In case of peptide GLKEGIPAL, an extensive hydrogen bond network is seen involving P9 leucine residue and residues of the heavy chain that line the binding grove including Asp76, Thr80, Tyr84, Thr143, Lys1467 and Trp147 (FIG. 2a. Upper panel). In contrast, for the extended peptide GLKEGIPALDN, the hydrogen bond between Lys146 and terminal carboxy group of P9 leucine is replaced with one between Lys146 and the P10 aspartate side chain, since Lys146 adopts a different orientation (FIG. 2a. Lower panel). In case of the peptide pair GLLPELPAV and GLLPELPAVGGNE, similar hydrogen bond network is observed for both peptides with only a minor difference in the crystal structure of the longer peptide. The hydrogen bond interaction between the terminal carboxylate of P9 valine and Lys146 is missing in the crystal structure with the longer peptide (FIG. 2b). The same is true for the peptide pair YLSPIASPL and YLSPIASPLLDGKSLR (FIG. 2c). As a result, the change in the orientation of Lys146 leads to the loss of hydrogen bond formation with the carboxylate of the P9 amino acid. However, hydrogen bond interaction between HLA-A*02:01 residue Trp147 and the backbone oxygen of the P8 amino acid remains conserved. Depending on the amino acid following residue P8 a novel hydrogen bond can be formed with the side chain of a compatible amino acid at P10 (here Asp10). Although, few residues in the longer peptides do not have detectable electron density in the crystal structure the peptide chain clearly projects into the solvent away from the binding pocket.

### Extending Peptides do not Significantly Destabilize HLA-A*02:01

To determine the relative stability of the individual HLA-A*02:01-peptide complexes the inventors followed their thermal denaturation by differential scanning fluorimetry. The melting temperatures (Tm) obtained from the melt curves allowed the inventors to compare the stability of the different complexes (FIG. 3). It was observed that complexes of HLA-A*02:01 with extended peptides are as stable as those with their equivalent nested peptides with not more than 8° C. difference between them. For example, the Tm for HLA-A*02:01 complex with GLKEGIPAL is 63° C. while that with its longer peptide counterpart is 61° C. Addition of 6 extra residues to peptide YLSPIASPLL also only changes the Tm of the complex with peptide YLSPIASPLLDGKSLR by about 7° C. [FIG. 3 and ref. (20)]. Interestingly, some of the complexes with nested peptides are as stable as those with longer peptides (compare GLKEGIPAL with GLLPEPPVGGNE, FIG. 3). It is also worth noting that there are some variations in the stability of complexes with different nested peptides. For instance, peptide GLKEGIPAL forms a less stable complex as compared to peptide GLLPELPAV (FIG. 3). Although most interactions between HLA-A*02:01 and the peptides are conserved there is a significant difference in the hydrogen bond interaction between Tyr84 with their terminal corboxylate (3.65 Å for G9L and 2.9 Å for G9V, FIG. 2). Without being limited to any particular theory, the lack of an intimate hydrogen bond interaction of the terminal amino acid with Tyr84 in G9L peptide is likely a major contributor to the reduced melting temperature.

### Lysine 146 Lift

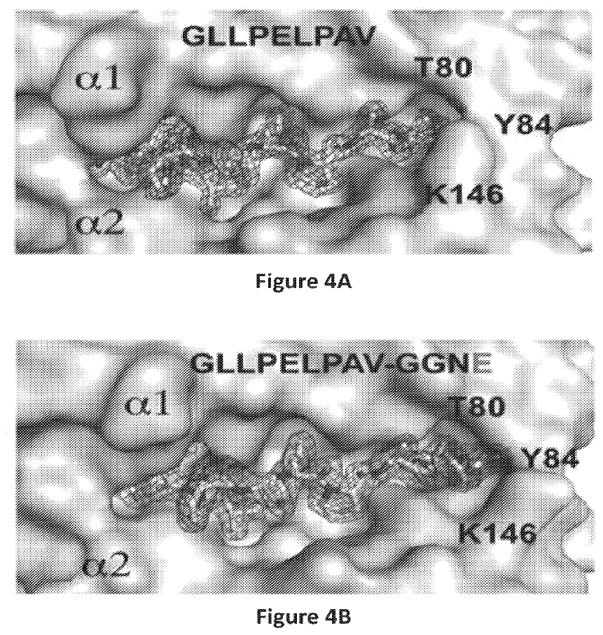

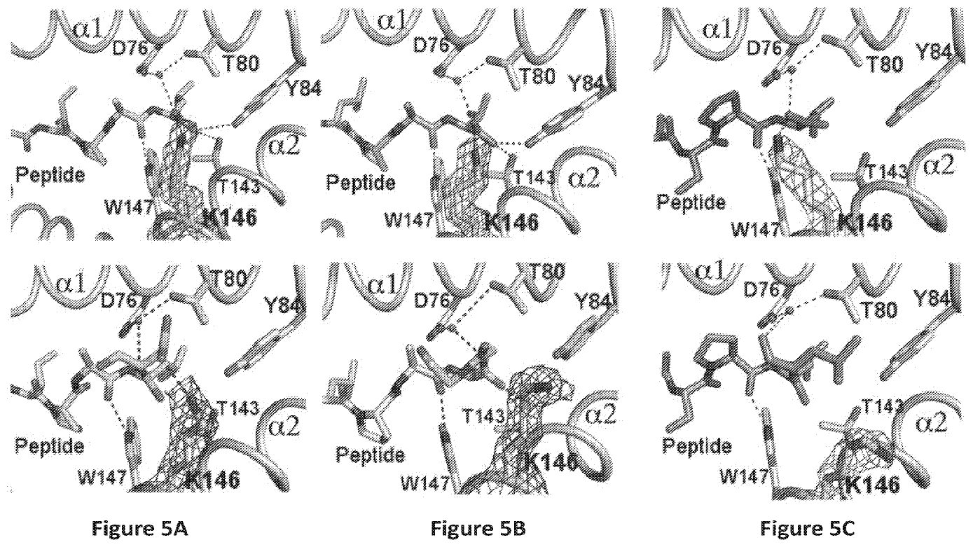

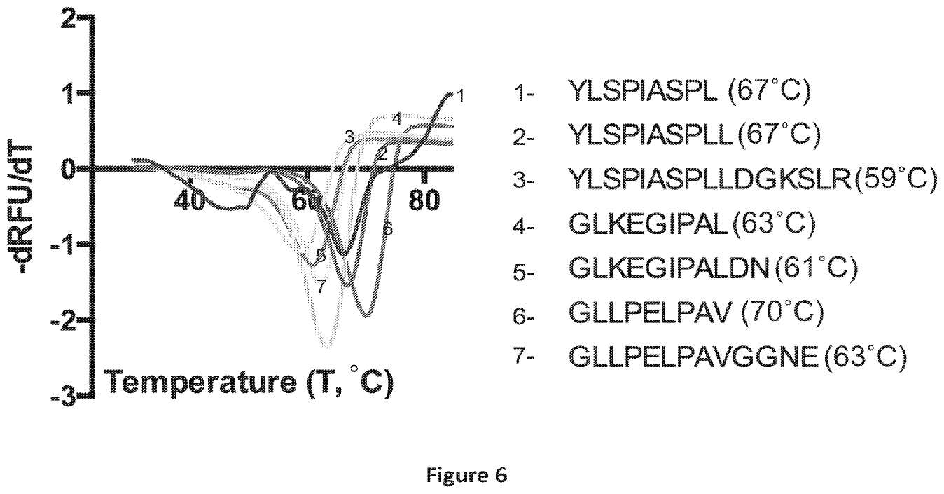

The different orientations of Lys146 upon binding of the longer peptides open the F pocket of HLA-A*02:01 and is required for the C-terminally extending amino acids to project into the solvent, since Lys146 forms a partial “lid” above the F pocket, held in position by the hydrogen bond interaction of the carboxylate of the C-terminal amino acid (P9) of any nested peptide (FIGS. 2 and 4). Even though, the position of Lys146 is not precisely conserved between the different structures of HLA-A*02:01 bound to the extending peptide, the lift of the residue to accommodate the peptide extension seems to be consistent. In case of the crystal structure of HLA-A*02:01 with YLSPIASPL, YLSPIASPLL, and YLSPIASPLLDGKSLR there are variations in the way that the two nested peptides are accommodated in the binding grove. With YLSPIASPL, the binding of the residues is quite conventional with the P2 and P9 anchor residues binding to the A and F pocket. Surprisingly, however, YLSPIASPLL, the nested peptide with one extra leucine residue at the C-terminal end of the peptide undergoes certain extent of bulging to accommodate the terminal leucine residue (P10, instead of P9) as the anchor residue in the F pocket (FIG. 4d). The difference in the binding of these two nested peptides to HLA-A*02:01 underscores the requirement for a sequence motif or particular amino acid features within the bound peptide to induce movement of Lys146 to open the F pocket. Since a mere increase in length of the peptide does not cause the change in orientation of Ly146, it is likely that the addition of charged residues within the C-terminal extension is the contributing factor to open the F pocket. Thus, in addition to the previously identified “Tyr84 swing” to accommodate the peptide FVLELEPEWTVK (UFP16-27), it was observed that a “Lys146 lift” in HLA-A*02:01 as a second mechanism of opening the F pocket induced by the extending peptides YLSPIASPLLDGKSLR, GLKEGIPALDN and GLLPELPAVGGNE (FIG. 5).

The addition of charged amino acids in the extended peptides, either directly follow the P9 residue or in up to three residues distance (FIG. 5c) of the nested peptide. The previously reported longer peptide contains a C-terminal addition of a positively charged residue (FVLELEPEWTVK) that opens the binding grove using the “Tyr84 swing” mechanism. Here, the inventors observed that longer peptides with a negatively charged residue within the C-terminal addition open the binding grove using the “Lys146 lift” mechanism. Thus, the opening of the binding grove of HLA-A*02:01 by longer peptides from T. gondii seems to be dictated by the charge of the first charged residue that follows the C-terminal amino acid of the nested peptide, since the peptide YLSPIASPLLDGKSLR also contains a lysine residue following the aspartate, but no “Tyr84 swing” was observed.

### DISCUSSION

αβT cell receptor (TCR) recognition of MHC presented microbial peptides initiates T cell mediated immunity against infection. Generally, the TCR binds with both TCR α and β chain in a diagonal orientation above the MHC molecule. While the germline encoded complementary determining region (CDR) 1 and 2 loops bind to MHC, the hypervariable loops CDR3α and 3β specifically bind and recognize the peptide and provide antigen specificity. Since MHC II has an open binding pocket and peptide ligands, typically 15-20 amino acids bind with both N- and C-termini hanging over the end of the groove, the precise presentation of the peptide extremities is not crucial for TCR recognition. However, MHC I has a closed binding groove and peptides bind with both ends tucked inside the binding pocket. Since MHC I presents peptides in a more confined space compared to MHC II, the TCR of CD8 T cells often contact and discriminate their entire peptide sequence. In the present invention, the inventors have focused on a panel of C-terminally extended peptides that contain a negatively charged amino acid within their C-terminal extensions. While these peptides contain a canonical HLA-A2*02:01 binding motif at their N-terminus, addition of the C-terminal extensions render any predictions about their binding to MHC I difficult. While the N-terminus of these peptides bind like canonical peptides to the MHC I allele HLA-A*02:01, the C-terminal extensions induce a structural change at the F pocket to allow their extension into the solvent. The inventors thereby show that only charged residues that follow the P9 amino acid of a canonical peptide within 3 or less amino acids induce the structural change that allows them to bind to MHC I and stabilize the complex.

While immunodominant T. gondii peptides that elicit protective CD8+ T cell responses are often derived from secreted proteins, such as Gra6, the extended T. gondii peptides can also be derived from cytoplasmic proteins. At present, it is not explicitly clear, how peptides are loaded onto HLA-A*02:01, whether their loading is TAP dependent, or why they are not trimmed to a shorter length in the proteasome or by the aminopeptidase ERAAP. It is interesting to note that other HLA alleles, such as HLA-A3 and HLA-A11 prefer peptide ligands that contain C-terminal lysine residues and TAP translocates preferentially peptides with basic (such as lysine) and hydrophobic C-termini. This demonstrates that TAP is both able to transport extended peptides across the ER membrane if needed and also illustrates that peptides ending with a positively charged amino acid are not necessarily trimmed further into shorter peptides by the aminopeptidase ERAAP.

TABLE 1

| | HLA-A*02:01 with peptide | | | | | | |
|---|---|---|---|---|---|---|---|
| | G9L | G11N | G9V | G13E | Y9L | Y10L | Y16R |
| Data collection | | | | | | | |
| Resolution | 50.0-1.85 | 50.0-2.30 | 51.6-1.86 | 40-2.10 | 50.0-2.5 | 50-2.7 | 50.0-2.4 |
| range (Å)^{a} | (1.89-1.85) | (2.38-2.3) | (1.89-1.85) | (2.18-2.10) | (2.59-2.5) | 2.8-2.7 | (2.44-2.4) |
| Completeness | 93.2 (96.1) | 96.6 (81.1) | 98.8 (97.5) | 99.6 (97.9) | 100 (100) | 93.4 (95.4) | 97.9 (85.2) |
| (%)^{a} | | | | | | | |
| Number of | 35,425 | 19,954 | 37,513 | 26,570 | 15,320 | 11,732 | 17,776 |
| unique | | | | | | | |
| reflections | | | | | | | |
| Redundancy | 2.7 | 3.4 | 3.7 | 3.6 | 3.7 | 2.8 | 3.4 |
| R_{sym} (%) | 8.6 (53.1) | 7.1 (33.5) | 7.8 (31.1) | 12.8 (57.0) | 16.1 (71.1) | 19.0 (66.4) | 13.5 (66.3) |
| Rₚᵢₘ (%) | 6.1 (38.5) | 4.5112 (21.7) | 4.7 (18.8) | 7.8 (37.7) | 9.7 (42.9) | 13.1 (45.4) | 8.4 (42.6) |
| I/σ^{a} | 21.5 (3.1) | 21.2 (3.3) | 20.3 (4.6) | 13.3 (2.1) | 7.9 (2.1) | 8.3 (2.4) | 11.3 (1.7) |
| Refinement statistics | | | | | | | |
| Number of | 33,590 | 18,963 | 35,290 | 25,169 | 14,464 | 11,305 | 16,851 |
| reflections | | | | | | | |
| (F > 0) | | | | | | | |
| Maximum | 1.85 | 2.3 | 1.86 | 2.1 | 2.51 | 2.7 | 2.4 |
| resolution (Å) | | | | | | | |
| R_{cryst} (%) | 20.8 (25.1) | 20.9 (36.9) | 20.9 (23.9) | 20.9 (30.8) | 19.9 (26.3) | 21.3 (24.3) | 20.4 (35.5) |
| R_{free} (%) | 24.4 (29.4) | 25.7 (34.1) | 23.4 (29.4) | 23.7 (31.3) | 27.5 (33.3) | 28.5 (32.9) | 25.8 (36.3) |
| Number of | 3371 | 3151 | 3397 | 3227 | 3170 | 3142 | 3210 |
| atoms | | | | | | | |
| Protein | 3047 | 3011 | 3060 | 3015 | 3012 | 2996 | 3032 |
| Peptide | 62 | 70 | 59 | 66 | 67 | 75 | 77 |
| Glycerol | 3 | 2 | 3 | 0 | 3 | 1 | 2 |
| Solvent | 241 | 57 | 280 | 143 | 71 | 62 | 83 |
| molecules | | | | | | | |
| (waters) | | | | | | | |
| Ion | | | | | | | 1 |
| Ramachandran | | | | | | | |
| statistics (%) | | | | | | | |
| Favored | 98.7 | 97.6 | 98.7 | 98.9 | 97.6 | 96.0 | 97.9 |
| Outliers | 0.0 | 0.0 | 0.0 | 0.0 | 0.0 | 0.0 | 0.0 |
| I. R.m.s.d. | | | | | | | |
| from ideal | | | | | | | |
| geometry | | | | | | | |
| Bond length | 0.0064 | 0.0075 | 0.0061 | 0.0076 | 0.01 | 0.01 | 0.009 |
| (Å) | | | | | | | |
| Bond angles | 1.12 | 1.22 | 1.11 | 1.22 | 1.45 | 1.46 | 1.33 |
| (°) | | | | | | | |
| Average B | | | | | | | |
| values (Å²) | | | | | | | |
| Protein | 29.6 | 48.5 | 15.1 | 27.5 | 26.3 | 20.8 | 36.9 |
| Peptide | 17.8 | 48.2 | 13.0 | 26.2 | 25.8 | 26.3 | 28.8 |
| Water | 29.4 | 41.7 | 24.8 | 26.7 | 21.0 | 15.8 | 35.2 |
| molecules | | | | | | | |

### Example 2

Design and Evaluation of HLA-A, -B, and -C Binding Peptides that Disrupt Inhibitory KIR/MHC Interaction and Activate NK Cells.

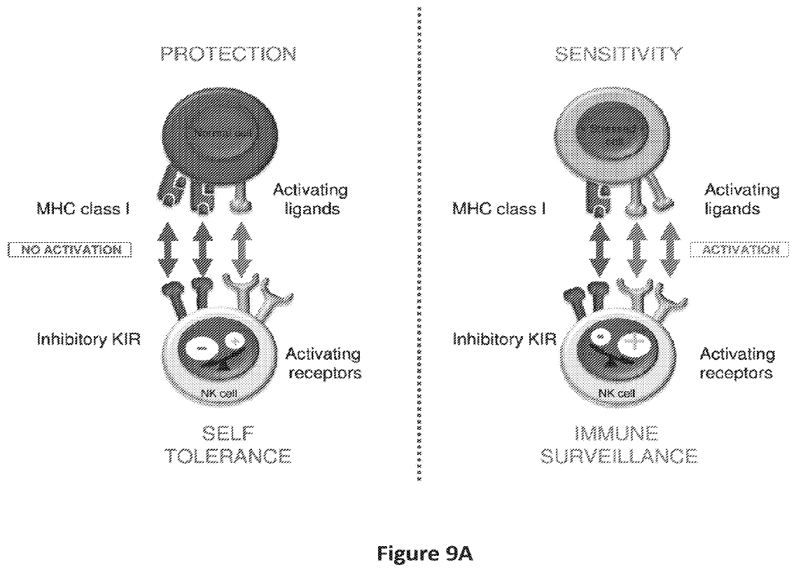

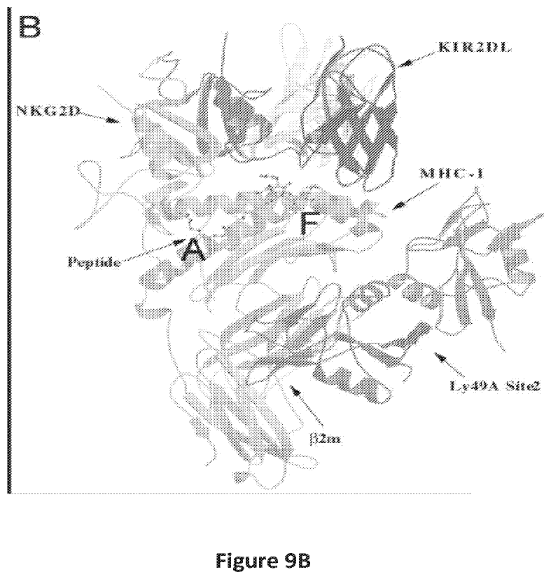

NK cells contribute about 10% of all circulating lymphocytes and are constantly patrolling. They are very effective in destroying virally infected or tumor cells. Unlike T cells, receptors on NK cell that engage MHC like molecules are far less specific for the presented peptide antigen and mainly bind MHC I itself. Inhibitory receptors on NK cells (KIRs, CD94/NKG2A) constantly screen for MHC I expression on APC's in an effort to detect down-regulation of MHC I by viruses, which evolved mechanisms to evade T cell recognition and killing. Activating receptors on the other hand, such as NKG2D engage non-classical MHC molecules that are up-regulated upon cellular stress or infection, such as RAE-1, H-60, MICA, MICB and ULBPs. In a healthy cell, interactions between inhibitory NK receptors dominate, leading to the inhibition or tolerization of NK cells against self. Upon infection, the balance between positive (activating receptors) and negative (inhibitory receptors) signals is changed in favor of NK cell activation, due to the up-regulation NK cell activating ligands, leading to NK cell activation and target cell killing (FIGS. 9A-9B).

NK cells and T cells recognize MHC class I molecules and orchestrate the cell-mediated clearance of an infected or tumor bearing APC. Recognition of an MHC I presented peptide by cytotoxic T cells leads to the killing of the infected APC, while activation of NK cells depends on the sum of activating and inhibitory signals, which are modulated by the expression of activating or inhibitory ligands on the APC. Since NK cells are important for the clearance of infection and the control of tumor progression or relapse after chemotherapy, activation of NK cells by disrupting the interactions between polymorphic inhibitory Killer Immunoglobulin Receptor (KIR) and their polymorphic HLA ligands will be beneficial for the survival of the host. It has been shown that disrupting the interaction between an inhibitory KIR with its MHC Class I ligand HLA-C*03:07 using the anti KIR2DL2 antibody IPH2101 shows promising efficacy against relapse of blood cancers, such as CML and AML after chemotherapy. IPH2101 is currently in phase II clinical trials. Since healthy cells usually express very few activating but many inhibitory ligands, such as MHC I (both classical and non-classical) NK cells are tolerized against these cells through inhibitory NK signals. Infection or cellular stress leads to upregulation of NK cell activating ligands, titling the balance in favor of NK cell activation. Since certain viruses and tumors can downregulate expression of activating NK cell ligands, releasing the brakes of inhibitory NK signaling is an alternative way of activating the NK cell. The inventors have discovered a novel mechanism by which MHC I presented peptides that are extended at their C-termini by up to 7 amino acids are efficiently bound by HLA-A*02:01 while inducing a structural change at the extremity of the peptide binding groove, the F pocket, of HLA-A*02:01 involving the evolutionary conserved residues Tyr84 and Lys146. This change may not affect recognition by most T cells, since the TCR binding site is more centrally located. However, inhibitory KIRs, which recognize HLA-A to -C alleles bind centered above the F pocket, suggesting that their binding will be disrupted. The discoveries herein allowed the inventors to design peptides for any given HLA allele.

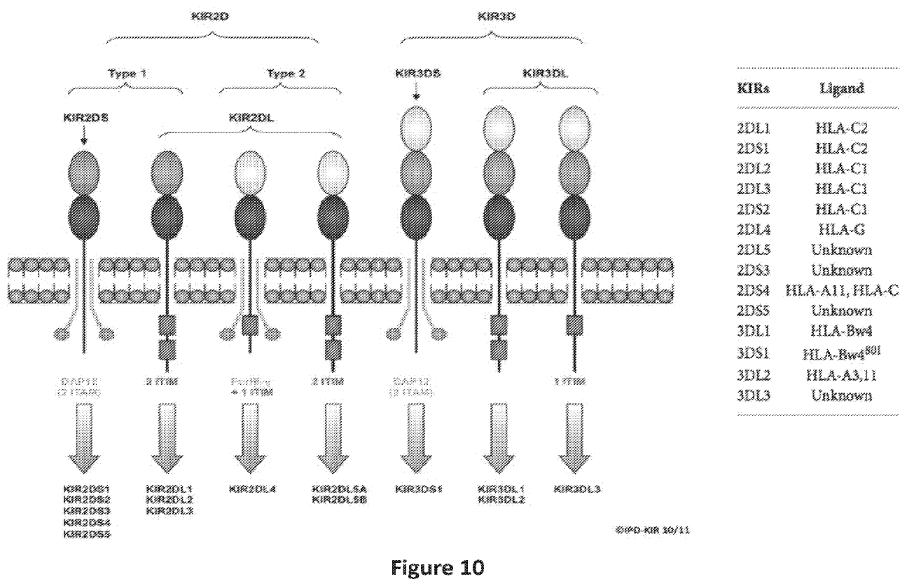

Killer Immunoglobulin receptors (KIRs) are a family of receptors expressed on NK cells, as well as on some T cell subset (28). They are composed of either two (KIR2D) or three (KIR3D) Ig domains and express either a short (S) or long (L) cytoplasmic tail. KIRS are activating receptors, since they can associate with DAP12, while KIRL are inhibitory receptors that encode ITIM motives in their cytoplasmic domains. Similar to its MHC I ligands, KIR receptors have multiple alleles and are highly polymorphic in sequence. Ligands for inhibitory KIRs are well defined, while ligands for KIRSs are largely unknown (FIG. 10). In viral infections, particular combinations of NK-activating receptors or KIR and their ligands are protective. For example, the presence of KIR3DS1 and its putative ligand HLA-Bw4 (180) was identified as key factor in preventing HIV infection to lead to full-blown AIDS. In hepatitis C infection, KIR2DL3 homozygosity and HLA-C1 homozygosity are beneficial in both early eradication of infection and response to standard treatment (type I IFN+ribavirin administration). Homozygosity of KIR2DL3 and HLA-C1 alleles has been reported to lead to lower levels of NK inhibition than other pairs of KIR ligand combinations (35, 36), suggesting that this underlies the enhanced response to hepatitis C.

NK cells are versatile killers, and their activation has clinical relevance for various diseases. Tumor infiltration of NK cells in humans may be associated with a better prognosis in various solid tumors, such as squamous cell, lung, gastric, and colorectal carcinoma. Especially tumors that downregulate classical MHC I expression to evade inhibitory KIR signaling, or upregulate non-classical MHC I activating ligands (altered self-recognition) are ideal targets for NK cells. NK cells were also shown to be effective in the eradication of established hematological diseases, such as acute myeloid leukemia (AML) multiple myeloid lymphoma (MML) and chronic myeloid lymphoma (CML). Recently, the administration of the anti-inhibitory KIR antibody IPH2101 to autologous multiple melanoma (MM) cells enhanced NK cell cytotoxicity against the tumor cell but not normal cells by blocking the interaction of KIR2DL1-3 with HLA-C. These data demonstrate that disrupting a single inhibitory KIR interaction with its MHC I ligand can lead to NK cell activation and tumor killing, while NK cell tolerance against normal cells in not impaired. The antibody IPH2101 is now in phase II clinical trials.

Peptides are designed having an MHC class I epitope and 1-7 additional amino acids where at least one of the additional amino acids bears a charged side group. Newly designed peptides are synthesized and tested for the ability to extend the C-terminus into the solvent by determining crystal structures of MHC class I molecules bound to the peptide. An example of the methodology is shown below.

The TCR DMF5 recognizes the tumor epitope (AAGIGILTV) of the MART-1 protein presented by HLA-A*02:01. Using Surface Plasmon Resonance (SPR), the designed variants AAGIGILTVK, AAGIGILTVD, or AAGIGILTVDGK are tested as to whether DMF5 TCR binding differs when HLA-A*02:01 is presenting the ligand AAGIGILTV or the designed variants.

Peptides were also designed and tested for HLA-B and HLA-C binding and solvent extension. HLA-B*57:01 is refolded and crystallized with the core self-peptide LSSPVTKSF, as well as the three variants containing the “D”, “K” or “DGK” amino acid additions at the C-termini. HLA-C*03:07 is refolded and crystallized with the core peptide GAVDPLLAL, as well as the three variants containing the “D”, “K” or “DGK” amino acid additions at the C-termini.

Peptides were also tested for the ability to disrupt KIR binding but retain TCR binding. The inhibitory NK receptors KIR3DL1 and KIR2DL3 are expressed in insect cells as Fc fusion proteins and are tested for their ability to bind the respective HLA molecules presenting the core and extending peptides using SPR.

Designed peptides are also tested for the ability to load onto HLA expressed on cells and activate NK cell killing.

HLA-A2*02:01 is expressed on the surface of K562 cells and the ability of the MART-1 peptide as well as extended peptides to bind to cell-surface expressed HLA when pulsed with the K562 cell is measured. MFI of TCR tetramer binding to the K562 cells is determined using flow cytometry (FCM).

If abrogation of KIR binding to HLA-peptides complexes is observed, extended peptides are loaded onto HLA-B*58:01 and HLA-C*03:07 and tested when expressed on 721.221 B cells. For this study, peptides are pulsed with the APC and incubated with YTS transfectants expressing the matching KIR allele. Expression of the HLA ligand, on the 721.221 cell sends an inhibitory signal to inhibit NK cell killing, while a structural change within HLA upon binding of an extended peptide will result in NK cell activation and target cell killing. As a control, the antibody IPH2101, which blocks KIR2DL1-3 receptors and leads to killing of the HLA-C*03:07 expressing target cell is used as a control in the same assay.

Studies herein demonstrate that all major HLA I alleles-A, -B, and -C show structural flexibility to open the F pocket. This is extremely important, since MHC I is thought to bind peptides within confined pockets. Peptides can be designed for any HLA allele to break confinement and disrupt inhibitory KIR binding leading to NK cell activation. Peptides are easier to design compared to antibodies that lack broad KIRDL specificity (e.g IPH2101 for KIR3DL1-3).

Some non-limiting examples of C-terminally extended peptides are shown in Table 2 which can be used to induce NK cell activity against T. gondii.

TABLE 2
T. gondii peptides
Core peptides
YLSPIASPL
YLSPIASPLL
FVLELEPEWTV
GLKEGIPAL
GLLPELPAV
C-terminally extending
YLSPIASPLLDGKSLR
FVLELEPEWTVK
GLKEGIPALDN
GLLPELPAVGGNE

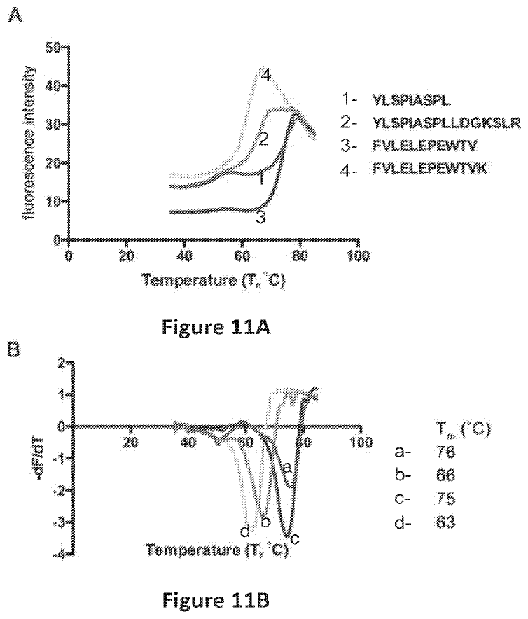

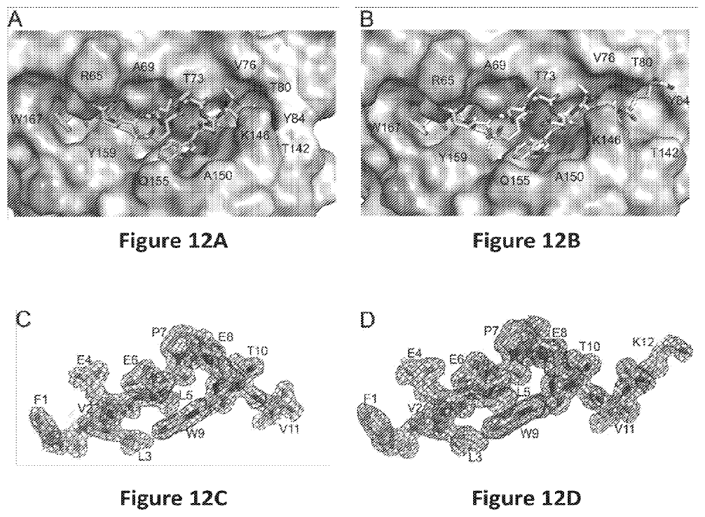

The extended peptides of Table 2 form stable complexes with MHC I based on thermal denaturation fluorimetry (FIGS. 11A-11B). Binding algorithms failed to predict binding of the longer peptides to HLA-A*02:01 (not shown).

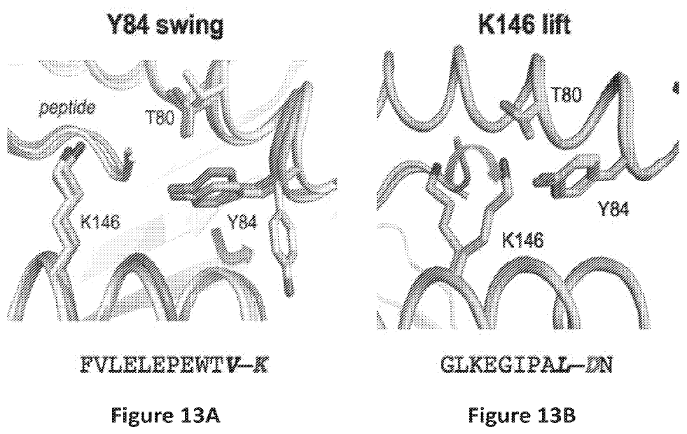

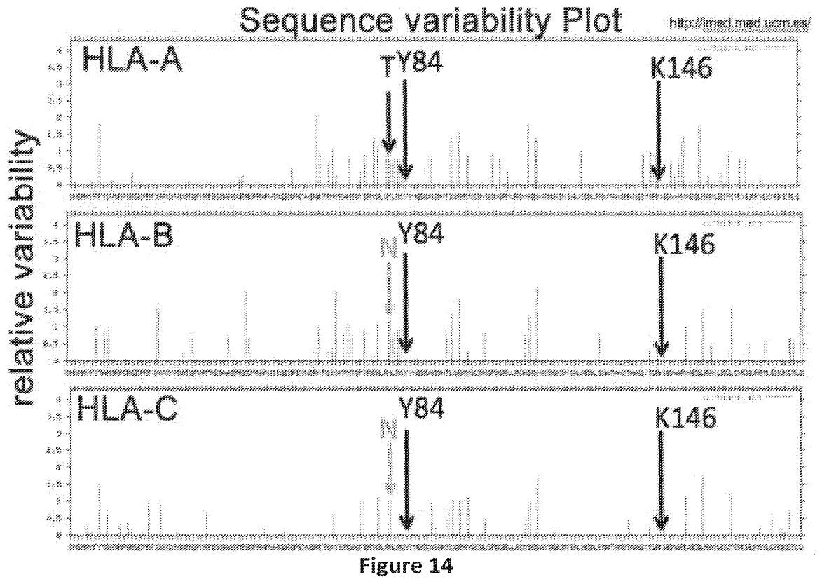

The crystal structures of all 9 peptides (Table 2) bound to HLA-A*02:01 was determined at a resolution between 1.6-2.6 A. The inventors noticed that while the core peptides bound as expected with the N- and C-terminal amino acid tugged in the MHC binding groove, the C-terminally extending peptides peptide induced a structural change at the F pocket of HLA-A*02:01. This structural change involved the residues Tyr84 and Lys146, which together form the lateral wall (Tyr84) and the lid (Lys146) to close the F pocket (see below). The peptide FVLELEPEWTVK that contains a positively charged lysine addition induced the “Tyr84 swing”, followed by a minor rearrangement of Thr80 (FIG. 11A-11D, FIGS. 13A-13B). Most extending peptides that were characterized, however, contained a negatively charged residue, either following the core peptide directly or with a few residues gap. Longer peptides with negatively charged additions did not alter the orientation of Tyr84, however, Lys146 moved up into the solvent (“Lys146 lift”) (FIG. 2). In summary, the inventors have identified two mechanisms of opening the F pocket of HLA-A*02:01, depending on the nature of first charged amino acid that follows the peptide binding core. Adding a hydrophobic amino acid, such as in the peptide YLSPIASPLL (core in bold), does not lead to the opening of the F pocket but and altered peptide binding compared to the core, in which residue P10 now is used as the anchor residue in the F pocket (FIG. 2, FIGS. 13A-13B).

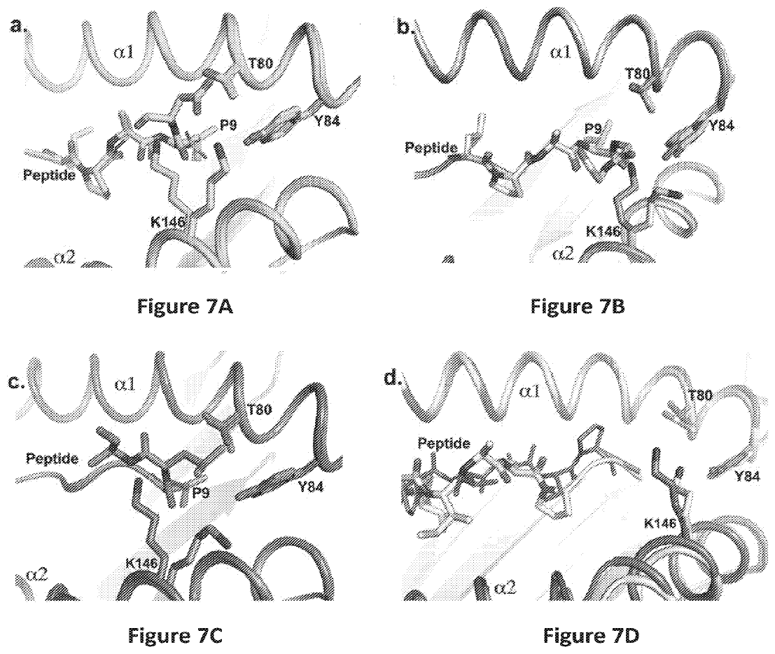

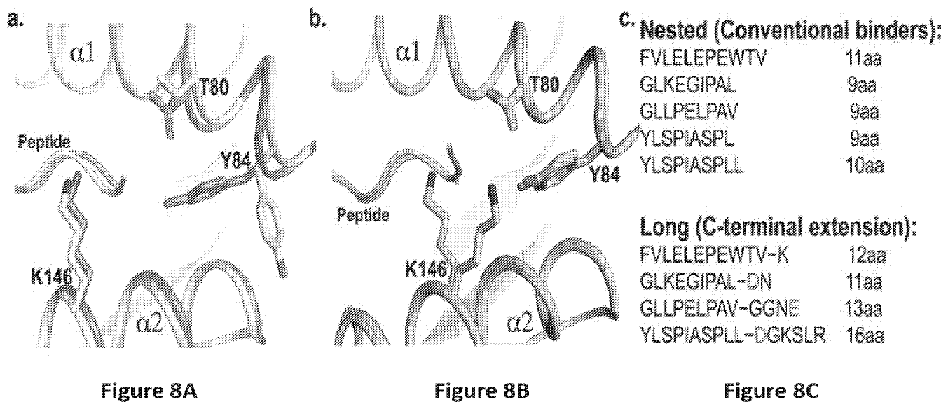

Since the residues Tyr84 and Lys146 are conserved across all HLA-A, -B, and -C alleles (FIG. 7), the opening of the gate of the F pocket was determined to be a universal mechanism that allows C-terminally extending peptides to protrude into the solvent. Since inhibitory KIRs bind above the F pocket of HLA-B and HLA-C and alanine scanning of Lys146 lead to loss of KIR binding, the interaction between inhibitory KIRs and the corresponding HLA allele is disrupted by this structural change. Current tools fail to predict binding of extended synthetic peptides to HLA-A*02:01.

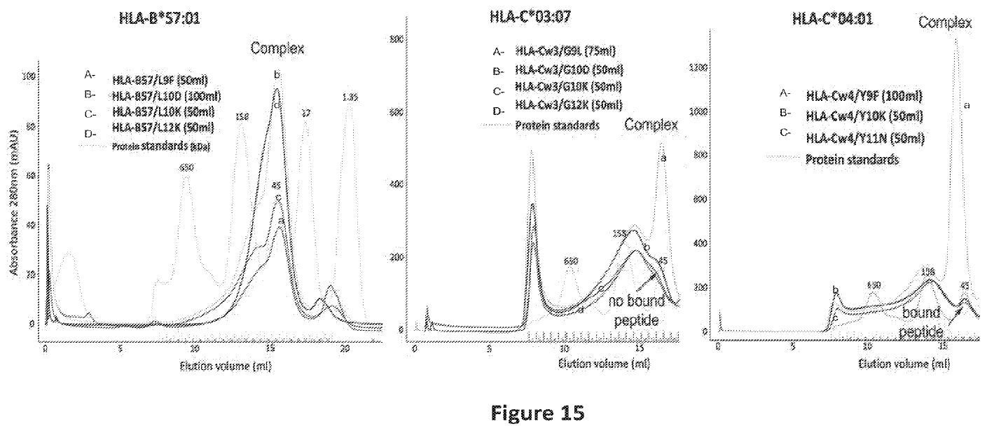

Core peptides were chosen that have been crystallized in complex with HLA-B as well as HLA-C and designed variants containing C-terminal extensions (Table 3). Detection of a protein peak corresponding roughly to the 44 kDa molecular weight marker on size exclusion chromatography and detection of the two corresponding bands on a SDS PAGE (˜33 kD for the heavy chain and ˜10 kD for β2M) suggests proper assembly of the heavy chain/β2m/peptide complex and demonstrate peptide binding. This indicated that the F pocket is flexible in these two representative alleles. Initial experiments suggested that HLA-B*57:01 refolds very well with both core and extending peptides (FIGS. 15A-15D). Also, a protein complex of proper size was observed by gel filtration and SDS PAGE for both core peptide G9L and at least on extending peptide (G10D) with HLA-C*03:07, although there appears to be a major contaminant band as well. HLA-C can be optimized while producing more HLA-B complexes for crystallization and structure determination using established methods. Crystallization is not major problem, since only HLA I-peptide complexes are crystallized and not ternary complexes containing a receptor. For HLA-A2*02:01, all 9 complexes were successfully crystallized thereby establishing a routine procedure. Structure determination is performed using molecular replacement (MR), using available reference structures.

TABLE 3
Designed peptides
HLA-A*02:01
G9V: AAGIGILTV
G10K: AAGIGILTVK
G10D: AAGIGILTVD
G12K: AAGIGILTVDGK
N10V: TNLVPMVATV
N13K: TNLVPMVATVDGK
HLA-B*57:01/HLA-B*58:02
L9F: LSSPVTKSF
L10K: LSSPVTKSFK
L10D: LSSPVTKSFD
L12K: LSSPVTKSFDGK
HLA-C*03:07:
G9L: GAVDPLLAL
G10K: GAVDPLLALK
G10D: GAVDPLLALD
G12K: GAVDPLLALDGK
HLA-C*04:01:
Y9F: YFDPANGKF
Y10K: YFDPANGKFK
Y11N: YFDPANGKFDN

Designed peptides are tested for the ability to disrupt KIR binding but retain TCR binding. The sequence of existing peptides, such as tumor derived peptides, is altered by adding the C-terminal extensions disclosed herein in an effort to allow TCR binding and tumor infiltrating lymphocyte (TIL) activation and tumor killing, while in addition disrupting inhibitory KIR binding to activate NK cells to provide additional killing. Real-time binding assays are used to measure the binding interaction between HLA-A and a representative TCR as well as HLA-B, and —C and allele matched inhibitory KIRs. The degree of TCR and KIR binding disruption is determined by observing the structural modification of the F pocket. While the TCR binding footprint on MHC I differs for each TCR most centrally binding TCRs should not be affected by this structural change. In contrast, KIR binding is more focused above the F pocket and should always be disrupted.

The well characterized TCR DMF5, which recognizes a tumor epitope of the MART-1 protein in the context of HLA-A2*02:01 is tested. The TCR from inclusion bodies is refolded, and measured for its binding to sensor chip immobilized HLA-A2-peptide complexes using Surface Plasmon Resonance (SPR). For this, HLA-A*02:01 is C-terminally biotinylated through incorporation of an avitag and immobilized on a streptavidin SPR sensorchip (CAP kit, GE Healthcare). In addition, the HLA construct is used to prepare TCR tetramers using any fluorophore-conjugated streptavidin for additional studies described herein.

Since TCR affinity for the decamer peptide (EAAGIGILTV) is roughly 7-fold higher (KD=5.6 μM versus 40 μM) than for the nonamer peptide (AAGIGILTV), the decamer peptide for the TCR tetramer staining is also tested. Refolding is carried out using standard protocols that have been widely used in my lab to generate other TCRs suitable for SPR or structure determination.

Synthetic DNA constructs of KIR3DL1 and KIR2DL2 are cloned and expressed as an Fc-fusion construct in SF9 insect cells. KIR-Fc proteins is immobilized on a anti human Fc capture chain and binding to refolded HLA-B*57:01 and HLA-C*03:07 is measured by SPR. HLA-core peptide ligands complexes are used as a positive control. Binding affinity of KR3DL1 to HLA-B*57:01 is of moderate affinity (KD=17 μM). Full abrogation of the KIR-HLA interaction is expected. Peptides are also designed peptides having longer C-terminal extensions (e.g DGK) with the expectation that the protruding peptide tail increases blocking of KIR binding. C-terminal extensions are extended based on the peptide sequences found in Table 2.

Peptides loaded onto HLA expressed on cells are tested for the ability to activate NK cell killing. Since HLA-A2*02:01 transfected K562 cells present exogenously pulsed peptides to CD8+ T cells, the synthetically designed peptides are tested in a similar assay. DMF5 TCR tetramers are prepared and their binding to HLA-A2*02:01 transfected K562 cells pulse with the MART-1 peptide as well as its variants is tested. K562 antigen presenting cells transfected with HLA-A2*02:01 were obtained. It has shown that when pulsed with exogenous peptides, CD8+ T cells specific for the pulsed peptide can be activated, suggesting they get sufficiently loaded onto HLA-A on the cell surface. These cells are pulsed with the designed synthetic peptides and tested by FCM, to determine whether the extending peptides prevent TCR tetramer staining. Further peptide processing is controlled using brefeldin A to block newly synthesized HLA-A2 translocation to the plasma membrane before peptide pulse to see whether these peptides can directly load to HLA-A2 on the cell surface. Alternatively, inhibitors of the proteasome are used to block peptide processing, while allowing the peptide to be loaded inside cellular compartments, if necessary. This experiment establishes that peptides can be loaded onto fully folded or nascent HLA-A2, which is prerequisite for the cell based killing assay described below.

The ability of KIR3DL1 or KIR2DL2 transfected YTS cells to kill HLA expressing 721.221 target cells is tested. Since YTS cells are devoid of any inhibitory KIR, expression of a single KIR will tolerize YTS cells against the 721.221 target cell expressing the allele-matched HLA-B*58:01 (instead of HLA-B*57:01 used for SPR and crystallography) or HLA-C*03:07. Established protocols are used. HLA-B*58:01 is identical in sequence in the binding region for KIR3DL1 and it is expected to bind KIR3DL1. Using this system the degree of inhibition is determined using different concentration of peptides. Target cell killing is assessed using a europium-based fluorescence killing assay. In this setup, the antibody IPH2101 serves as a positive control for killing, and the efficacy of extending peptides to induce killing is compared. The inventors express IPH2101, for which the sequence is available (International Patent Application No.: PCT/US2013/063068), similar to Fc fusion proteins in insect cells. Without being limited to any particular theory, the efficiency of peptide loading to HLA may determine the total number of killed cells.

### Example 3

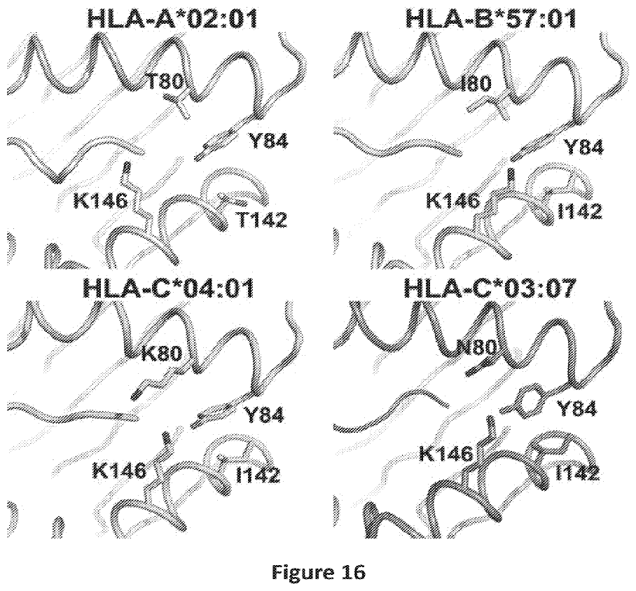

A structure-based sequence comparison of these HLA molecules was performed to identify possible amino acid differences between these alleles that cluster around the F pocket. Two amino acids are located around the F pocket at position 80 and 142 that differ between HLA-A*02:01, HLA-B*57:01, HLA-C*03:07, and HLA-C*04:01 (FIG. 16). Amino acids that are located underneath the peptide, at the bottom of the binding pocket in this analysis, were not included since they provide specificity for the presented peptide. While comparing these structures, it was noted that all alleles contain different amino acids at position 80. Thr80 (HLA-A), Ile80 (HLA-B,) Lys80 (HLA-C*04:01), and Asn80 (HLA-C*03:07). It is believed that the nature of the amino acid at position 80 can influence the F pocket flexibility. In addition amino acids at position 142 also differ between HLA-A*02:01 (Thr142), while all other alleles have Ile142.

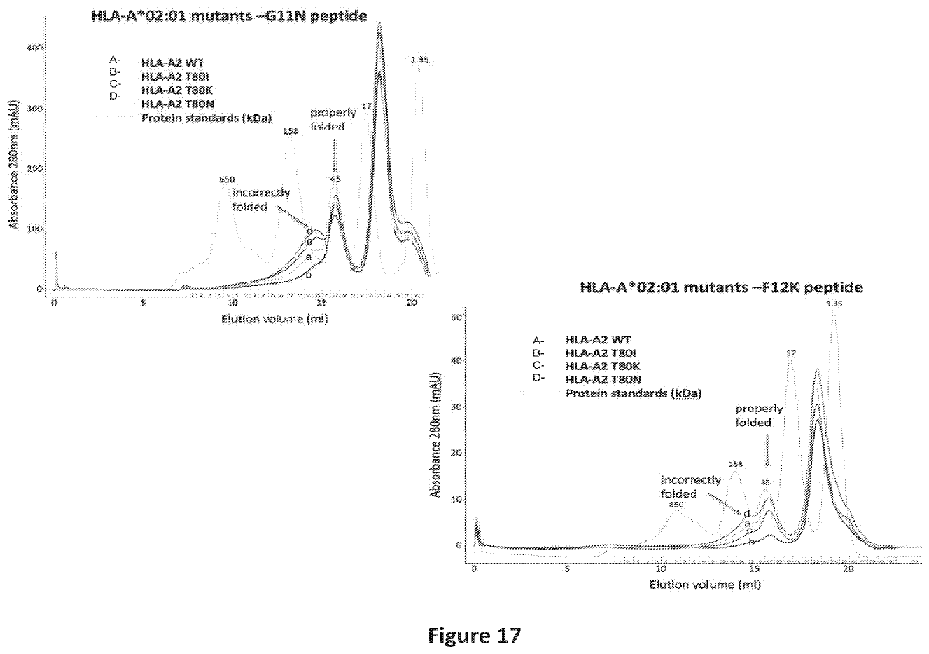

The inability of HLA-C*03:07 to refold may be a consequence of Asn80, which is bulkier than Thr80 and less flexible compared to Lys80. Although it does not appear to engage in hydrogen bond interaction with Tyr84 and Lys146 in the structure shown in FIG. 16, it has the ability through its polar nature to interact and stabilize both residues in solution. Although Lys80 found in HLA-C*04:01 is bulkier compared to Asn80, it may very well undergo a “Lys80 lift” upon binding of the extending peptides. This “altered” structural change of the F pocket would increase the understanding of F pocket flexibility. From the structural comparison and the refolding studies, the inventors have found that amino acid 80 primarily influences F pocket flexibility but whether amino acid at position 142 also modulates the F pocket flexibility is not clear yet. It is possible that replacing T80 with amino acids Ile, Lys, and Asn as found in other common alleles may affect refolding. Preliminary data using HLA-A*02:01 suggests that position 80 affects the refolding efficiency with extending peptide G11N, which induces the “lysine 146 lift” (FIG. 17). When all mutants were refolded following identical conditions and run over a size exclusion column without adjusting the chromatograms in any way, an increase in a species of not properly folded HLA/b2M complexes was observed. The inventors have identified that this population contains disulfide-linked dimers and trimers of the HLA heavy chain. Using the increase in incorrectly folded HLA-A*02:01 heavy chain as an indicator of impaired refolding, the refolding efficiency decreases with the following amino acids (found in the following alleles): Ile80 (HLA-B)>Thr80 (HLA-A)>Lys80 (HLA-C*04:01)>Asn80 (HLA-C*03:07). Similar results are obtained when refolding with the peptide F12K, which induces the “Tyrosine 84 swing”, although the overall protein concentration of the T80I mutant was low and could indicate a technical problem during concentrating the sample for the SEC analysis (FIG. 17). This suggests that both peptide induced structural changes favor a similar amino acid signature motif around the F pocket. The mutational data of HLA-A*02:01 correlates well the refolding abilities of the other alleles. HLA-C*03:07 has N80 and does not refold with extending peptides. However, there may be an influence on position 142 in F pocket flexibility, since refolding of HLA-A*02:01 was only impaired with the Asn80 mutation but not fully abrogated as compared to HLA-C*03:07. For example, HLA-A*02:01 may compensate for the bulkier amino acids at position 80 (Lys, Ile, Asn) by having a less bulky amino acid at position 142 (Thr). Whether a flexible F pocket in HLA-C*03:07 can be created was tested by exchange against the amino acids from HLA-A*02:01, either Thr80 or the combination of Thr80 and Thr142 together. Other amino acids at position 80 or mutate other alleles at position 80 and 142 were introduced as necessary.

### Example 4

Crystallization of HLA-B*57:01, HLA-C*04:01, and Selected HLA I Mutants with Core and Extending Peptides.

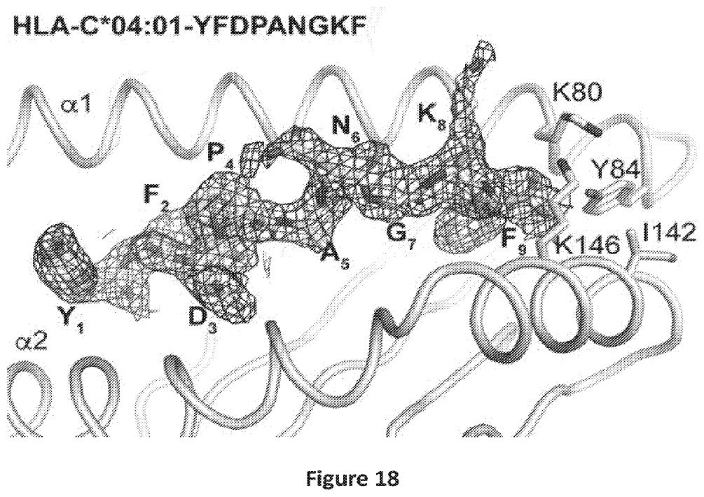

Successful refolding of HLA alleles with extending peptides may not provide sufficient proof that the F pocket of other HLA-B and -C haplotypes open similarly to HLA-A*02:0.1 Using protein crystallography it is shown that the structural adaptations and mechanisms that allow HLA-B*57:01 and HLA-C*04:01 to bind to extended peptides containing both negative and positive amino acids. HLA-C*03:07 mutant that has gained the ability to refold in the presence with extending peptides is crystallized. Collectively, these structures allow to establish the structural basis of the F pocket flexibility and/or altered modes of extended peptide binding. With this structural information, it is possible to predict which alleles have a flexible F pocket by multiple sequence alignment of the IMGT/HLA database (https://www.ebi.ac.uk/ipd/imgt/hla/). HLA I-peptide complexes are crystalized and not ternary complexes containing a receptor. Structure determination is performed using molecular replacement (MR), using the available reference structures (46-48). For HLA-A2*02:01, 9 complexes were crystallized. In addition, HLA-C04:01 with a high affinity core peptide (Y9F, YFDPANGKF, Table 2), that was associated with HLA-C*04:02 both in the breast cancer cell line MDA-231 and the ovarian cancer cell line UCI-107 (50), has been crystallized. The structure was determined at 2.05 Å and shows well-ordered electron density for the peptide Y9F bound between the α1 and α2 helices (FIG. 18). Electron density for Lys80 was disordered at the end, suggesting flexibility of Lys80 and supporting the hypothesis that Lys80 may directly open or assist in opening the F pocket as discussed above. Small crystals of HLA-B*57:01 with all 4 peptides (see Table 2) have also been obtained. The crystal morphology is the same for all crystals, suggesting that crystallizing extending peptides is no more difficult than that of the core peptide bound to HLA-B*57:01.

The structural and mutational studies coupled to a through sequence analysis provide an understanding of the sequence requirements that allow HLA class I molecules to open their F pocket. These results provide an understanding of the breath of this structural flexibility across the classical HLA I alleles.

### Example 5

### Studies of Designed Peptides for Retaining TCR Binding and Modulating KIR Binding.

It is believed that existing peptides, such as tumor derived peptides, can be modified by incorporating C-terminal extensions in an effort to disrupting inhibitory KIR binding (and activate NK cells), while at the same time TCR binding and tumor infiltrating lymphocyte (TIL) activation is unchanged. This is understood to result in the activation of both TILs and NK cells to simultaneously attack the tumor, increasing the anti-tumor efficacy over currently existing approaches that target only one immune cell type. To test this, real-time binding assays is used to measure the binding interaction between HLA-A*02:01 and the representative TIL TCR MART-1. It is determined to what degree (if any) TCR binding is affected by the structural alteration of the F pocket. While the TCR binding footprint on MHC I differs for each TCR, most of the TCRs bind centrally across the binding groove and expected to be unaffected by this structural change (5). In contrast, KIR bind above the F pocket and is expected to be impacted by changes around the F pocket. While much is known about inhibitory KIRL binding to their HLA class I ligands, activating receptors (KIRS), their HLA I restriction and/or peptide specificity are far less understood, even though the extracellular domains are highly similar (97% identity between KIR3DL1 and KIR3DS1). The impact of the peptide induced structural change of the F pocket in binding to both KIR3DL1 and KIR3DS1 is studied, since both bind to of HLA-B*57:01 (180) and the binding is influenced by the peptide sequence (51). The structural change of the F pocket is expected to abolish KIR3DL1 binding, while the impact of KIR3DS1 binding is not known. However, if KIR3DS1 is less sensitive to structural alterations of the F pocket, this would be beneficial for a subsequent immune response since it would tip the balance between inhibitory and activating NK signals toward NK cell activation.

### Example 6

### TCR Binding of HLA-A*02:01 Presenting Extended Peptides Using SPR

The well characterized TCR DMF5 was chosen, which recognizes a tumor epitope of the MART-1 protein in the context of HLA-A2*02:01. The TCR from inclusion bodies are refolded, and its binding to HLA-A*02:01-peptide complexes is measured using Surface Plasmon Resonance (SPR). For that, HLA-A*02:01 is enzymatically biotinylated at the C-terminus through incorporation of an avitag and immobilized on a streptavidin SPR sensorchip (CAP kit, GE Healthcare). Since TCR affinity for the decamer peptide (EAAGIGILTV) is roughly 7-fold higher (K_{D}=5.6 μM versus 40 μM) than for the nonamer peptide (AAGIGILTV) (4), the decamer peptide is also tested for the TCR tetramer staining. Refolding is carried out using standard protocols that have been widely used to generate other TCRs suitable for SPR or structure determination (52-57). Binding of this TCR clone is expected to be unaffected by the C-terminal amino acid addition, since the crystal structure of the ternary complex suggests that the peptide C-terminus is not involved in TCR recognition. The SPR assay is not always ideal when working with affinities that are in the 10 μM range, because of an increase of non-specific background binding of a highly concentrated TCR solution (˜100 μM is ideal to measure a 10 μM interaction). Therefore, TCR tetramers are generated from unconjugated streptavidin for SPR analysis. The tetra-valency is expected to increase the apparent TCR binding affinity dramatically, since 4 TCR monomers can simultaneously bind to 4 separate MHC I molecules immobilized on the chip, if the MHC I density is sufficient to allow for simultaneous binding. TCR tetramers are used, when the affinity is either greatly reduced or issues with non-specific background binding are encountered.

### Example 7

### Studies of KIRL Binding Using Extended Peptides.

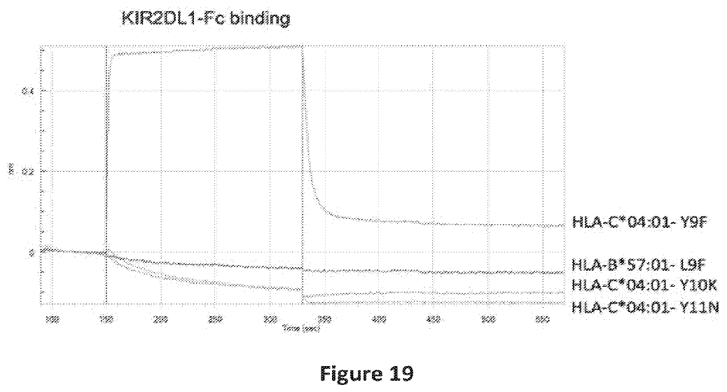

Synthetic DNA constructs of KIR3DL1 (HLA-B*57:01) and KIR2DL1 (HLA-C*04:01) are cloned and expressed as Fc-fusion constructs in SF9 insect cells. HLA-B*57:01 and HLA-C*03:07 are refolded with both core and extending peptides and captured on a Strepavidin sensor chip for SPR analysis or a SA sensor tip for BLI analysis as for HLA-A*02:01. Binding affinity of KR3DL1 to HLA-B*57:01 had previously been determined to be of moderate affinity (K_{D}=17 μM) (46). KIRDL-Fc fusion proteins are, therefore, used to measure the binding affinity, since their bivalency will increase overall binding affinity. This allows a more robust measure of whether the binding affinity is drastically reduced when HLA presents extended peptides, or simply reduced to a point where this assay is not reliable to measure the interaction, due to issues related to non-specific binding. Further, KIRDL-Fc fusion proteins are immobilized using an anti-human Fc antibody capture chip (GE Healthcare) and the binding of HLA-tetramers to the immobilized KIRDL receptors measured. This increases affinity even further (tetra-valency versus bivalency). Biotinylated HLA-B*57:01-L9F, HLA-C*04:01-Y9F, HLA-C*04:01-Y10K, and HLA-C*04:01-Y11N complexes were immobilized on a SA sensor tip and the binding of a 2 μM solution of KIR2DL1-Fc to each complex was measured. The C-terminally extended peptides all disrupt KIR2DL1 binding, regardless of whether they induce the “lysine lift” or the “tyrosine swing” (FIG. 19). HLA-C*04:01 refolded with the core peptide Y9F gives a strong binding signal to KIR2DL1, while the allele-mismatched HLA-B*57:01 presenting the core peptide L9F does not bind to KIR2DL1 as expected. This is proof of concept that structural alterations around the F pocket of HLA alleles that form the primary binding site for KIRL abrogate receptor binding. Next this study is repeated with a higher concentration of KIR2DL1-Fc to demonstrate that there is no low affinity binding to HLA-C*04:01 presenting extending peptides.

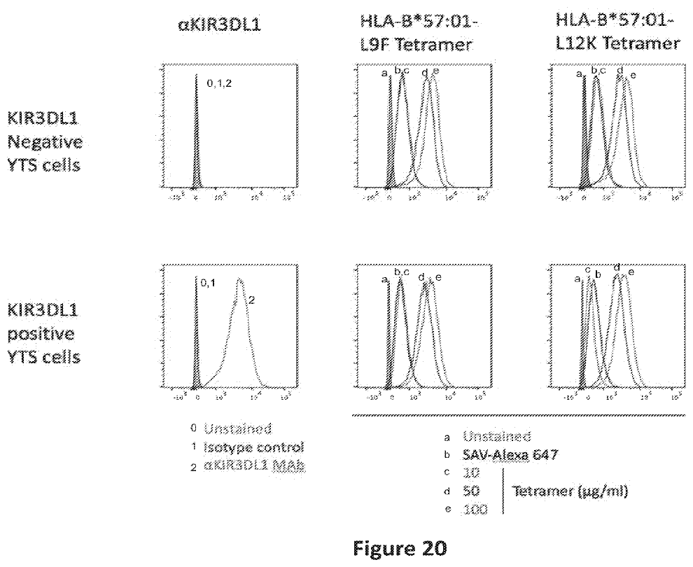

In parallel, whether the HLA-tetramers can stain K562 cells that have been transfected with the corresponding allele-matched KIRDL receptor is tested. Without being limited to any particular theory, it is expected that HLA tetramers presenting the core peptide will stain K562 cells, while staining with tetramers presenting the C-terminally extended peptides is greatly impaired and will overlap with the isotype control. Appropriate anti KIR antibodies are used as positive control (Anti-KIR3DL1 clone DX9 and anti-KIR2DL1 clone HP-MA4 from Biolegend). If low affinity KIR binding is observed (which is unlikely based on FIG. 19) peptides that contain longer C-terminal extension (e.g DGKSLR, see FIG. 2 can be used with the expectation that the protruding peptide tail will increase blocking of KIRL binding. In a preliminary study, YTS cells were used. KIR3DL1 expressing YTS cells (YTS-3DL1, 1×10⁵ total) were incubated with 10, 50 and 100 jag/ml of Alexa Fluor-647 conjugated HLA-B*57:01/L9F or HLA-B*57:01/L12K tetramers (or Streptavidin Alexa-Fluor-647 alone) for 30 min on ice and washed 2 times in PBS 2% FBS 0.01% sodium azide. Data were collected on a LSRII flow cytometer (BD biosciences) and analyzed using FlowJo software (Tree star). KIR3DL1 expression on YTS-3DL1 cells was verified using a PE conjugated anti-KIR3DL1 antibody or isotype control. Surprisingly, however, both tetramers bound to YTS cells, regardless whether KIR3DL1 was expressed or not (FIG. 20). Without being limited to any particular theory, this suggests that the tetramers bind to another receptor expressed on YTS cells, such as 2B4 and that binding to this NK receptor is independent of the structural change around the F pocket. 2B4 has been shown to be expressed on YTS cells (58).

### Example 8

### Studies of KIRS and the Structural Change Around the F Pocket.

A KIR3DS1-Fc fusion protein is generated and it's binding to HLA-Bw4 (180) (also known as HLA-B*57:01), which is refolded with core or extending peptides, is tested as discussed above. It is believed that there are differences in binding of KIR3DL1 and KIR3DS1 to HLA-B*57:01 and that the activating receptor is either insensitive to the presentation of extending peptides or has enhanced binding affinity to the extending peptide. If it is observed that KIR3DS1 binds to HLA-B*57:01 when presenting extended peptides, corresponding tetramer will be prepared, as well as control tetramers using the core peptide and analyze NK cells from PBMCs of allele-matched healthy donors for the KIR3DS1 expression. To what extent KIR3DS1 expressing NK cells have overlapping binding profiles with both tetramers or whether there are certain KIR3DS1+NK cells that differ in their binding ability to both tetramers, is determined. This sheds light onto the influence of the structural alteration of the F pocket in activating KIR3DS1+NK cells and hint at a possible role on the structural change in a normal immune response toward infection. Since extending peptides have been originally derived from T. gondii infected THP-1 cells, their recognition by KIRS receptors would confer protection. Since various combinations of KIRS receptors and HLA-I alleles confer protection against viral infections, it is believed that rather than recognizing a virus specific core peptide epitope, KIRS more generally “sense” the structural alteration of the F pocket by C-terminally extending microbial peptides.

### Example 9

Studies of Activating and Inhibiting KIR and Tetramers with Core and Extending Peptides.

20 HLA-B*57:01 positive samples have been identified and the staining of NK cells with HLA-B*57:01 tetramers containing both core and extending peptides is analyzed. While tetramers are generally used to stain T cells, the binding affinity of KIR3DL1 to HLA-B*57:01 (K_{D}=171M, (46)) is in range of typical TCR-pMHC I binding affinities (1-100 μM) and tetramers should stain NK cells equally well, similar to the presently disclosed HLA-B*57:01 tetramer staining of KIR3DL1 expressing YTS cells, regardless of whether they bound to KIR3DL1 or 2B4 in this assay (FIG. 20). Primary rhesus NK cells have also been successfully stained with a Mamu-A*02 peptide tetramer via binding to KIR3DL05 (59). Tetramers are prepared but using fluorophore conjugated streptavidin for subsequent flow cytometry study. Both NK cell numbers that are stained by the tetramers, as well as co-staining of NK cells are assessed to see whether there are any differences in the NK cell populations that bind to the two different tetramers. Since binding of KIR3DL1 is expected to be similarly abrogated by the extending peptides as compared to KIR2DL1 (FIG. 19), NK cells that retain staining likely express activating KIRS receptors. This is being tested with the anti-KIR3DL1 antibody (Biolegend clone DX9) that binds KIR3DL1 and the anti-KIR3DS1 specific antibody LS-C165538 (LSBio, Inc.). NK cell markers, such as CD56 and CD16, are included to differentiate less mature (CD56 bright, CD16-) and more mature (CD56 dim, CD16+) NK cells. The latter population is more commonly found in peripheral blood.

C-terminally extended peptides are expected to fully abrogate KIRL binding. Very high affinity binding reagents (e.g tetramers) are used to detect any residual low affinity binding. These results are important to assess a potential therapeutic value of the designed peptides. If the structural change around the F pocket is not sufficient to fully disrupt KIRL binding, the C-terminal extensions can be lengthened further in order to block KIRL-MHCI engagement directly by the C-terminally extending peptide tail. Also, a switch from KIR transfected YTS cells to K562 cells is made to overcome the off-target binding effect of the tetramers.

### Example 10

### Studies of Designed Peptide Presentation to Activate NK Cell Killing and Maintenance of Polyclonal T Cell Responses.

While the designed peptides refold with HLA-A, -B, and —C, it is not currently known how efficiently they are loaded onto HLA I molecules expressed on the surface of 721.221 cells. For T2 cells, which are deficient in a peptide transporter involved in antigen processing (TAP), the cell surface presentation of tumor associated peptides is dependent on the peptide concentration (60). Ten to 50 peptide epitopes could be detected on T2 cells when pulsed with peptide concentrations between 10⁻⁹ and 10⁻¹¹ (61). Since 721.221 cells are not deficient in TAP, 721.221 cells transfected with the TAP inhibitor ICP47 are included to increase loading efficiency. In the absence of TAP activity, exogenously pulsed peptides non-competitively load onto cell surface HLA molecules and enhance cytotoxicity of KIR transfected YTS cells. Two different HLA alleles (HLA-B*57:01 and HLA-C*04:01) are used in these studies to more generally assess whether efficient peptide presentation of core and C-terminally extended peptides is dependent on the HLA allele. In addition, whether polyclonal T cell responses are maintained using the designed peptides is tested, since any change in the TCR affinity toward the MHC I presented peptide antigen can impair development of this particular T cell clone. Positive selection of T cells with a reduced binding affinity could be impaired and could result in a less polyclonal T cell pool.

Adenoviral vector expressing the inhibitor ICP47 is used to transduce K562 cells. This study will establish that peptides can be loaded onto fully folded or nascent HLA-A2, which is prerequisite for the cell based killing assay below. In addition, using TAP deficient cells allows more precisely titration of the peptide to determine what concentration is sufficient to activate NK cell killing. By comparing the MFI between core peptides loaded in the presence or absence of the TAP inhibitor ICP47, the loading efficiency of peptides pulsed to APCs is assessed, and also whether extended peptides have a reduced loading efficiency is compared.

### Example 11

### Studies of Extended Peptides in Polyclonal T Cell Responses.

The peptide “TNLVPMVATV” is a well characterized epitope derived from the 65 kDa phosphoprotein (pp65) from Human cytomegalovirus (HCMV). Pp65 is targeted by 70% to 90% of HCMV-specific CD8 T cells (63, 64). This core peptide (T10V), as well the designed extended version T13K (TNLVPMVATVDGK, Table 3) is used for HLA-A*02:01 tetramer preparation. Tetramers of HLA-A*02:01-T10V have already been tested in flow cytometry of human PBMCs and are also available through the NIH tetramer core facility. The magnitude of CD8 T cell responses is analyzed by co-staining with both tetramers, which is conjugated with different fluorophores, such as AF647 and AF488. If necessary, the complete T10V specific CD8 TCR repertoire, or the population (if any) of CD8 T cells that are not stained with the extending peptide containing tetramer using ImmunoSeQ™ Assay (Adaptive Biotechnologies) can be analyzed. This assay uses 54 forward V gene primers and 13 reverse J gene primers, which are employed in a bias-controlled multiplexed PCR reaction to amplify the variable region of TCRβ chains. Synthetic control templates can be spiked into each sample, thereby enabling quantitation of input TCRβ templates from the read counts.

### Example 12

### Studies of Extended Peptides Presented by HLA Alleles.

The ability of KIR3DL1 or KIR2DL1 transfected YTS cells to kill HLA expressing 721.221 target cells is tested. Since YTS cells are devoid of any inhibitory KIR, expression of a single KIR will tolerize YTS cells against the 721.221 target cell expressing the allele-matched HLA-B*58:02 (instead of HLA-B*57:01 used for SPR and crystallography) or HLA-C*04:01. HLA-B*58:02 is identical in sequence in the binding region for KIR3DL1 and reported to bind KIR3DL1 allele. 721.221/HLA-C*04:01 cells expressing a TAP inhibitor as well as the corresponding KIR2DL1 YTS transfectants have been obtained. In these 721.221 cells, peptides have to be loaded on the empty HLA-C on the cell surface. The advantage of this system is that the degree of inhibition can be titrated using different concentration of peptides. Target cell killing is assessed using a europium-based fluorescence killing assay as described (67). In this setup, the antibody IPH2101 serves as a positive control for killing, and the efficacy of extending peptides to induce killing is compared. IPH2101 is expressed, for which the sequence is available (Patent application: PCT/US2013/063068), similar to Fc fusion proteins in insect cells. It is assumed that the efficiency of peptide loading to HLA will determine the total number of killed cells.

It has been shown that rhesus primary NK cells degranulate when incubated with 721.221 target cells expressing the allele matched MHC I molecule (59). NK cell cytotoxicity studies are expanded to include purified human NK cells, if necessary, using MHC I expressing 721.221 cells.

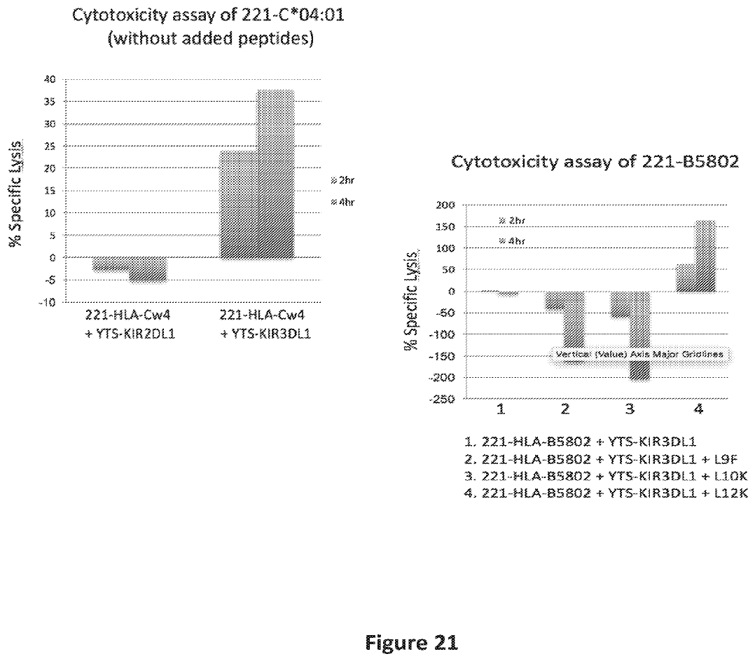

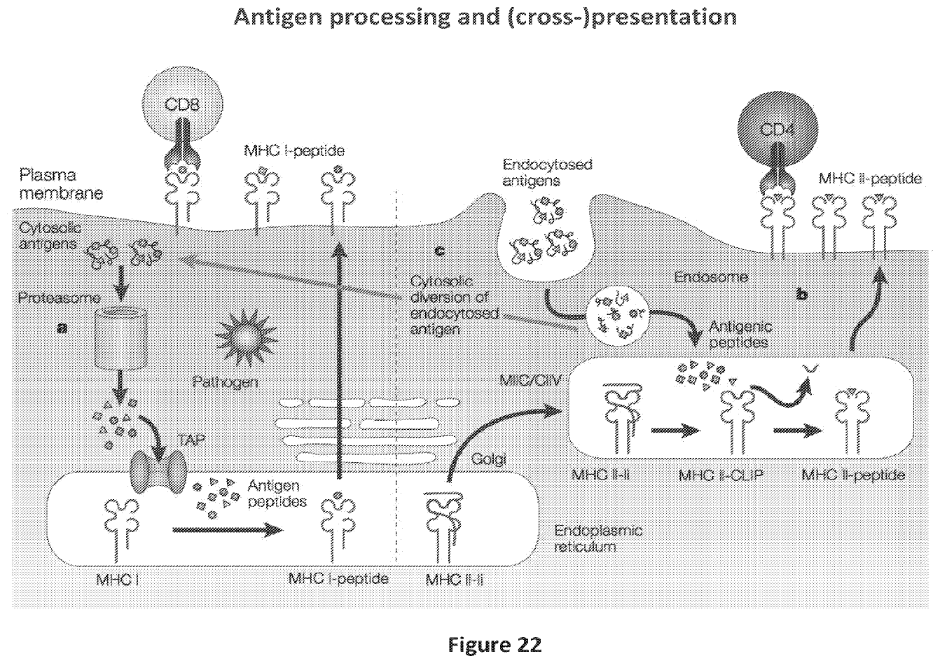

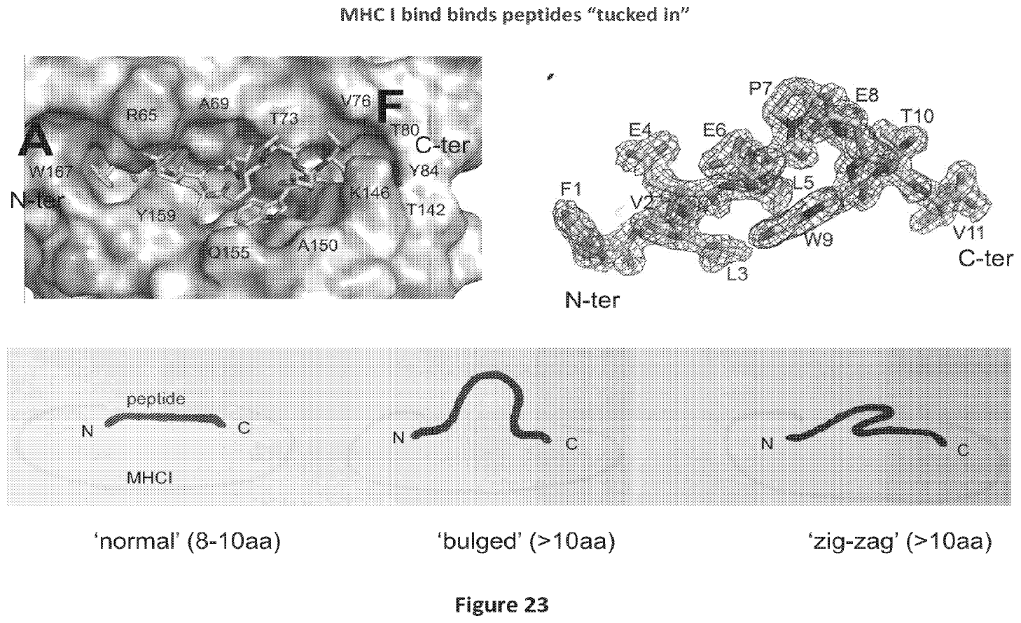

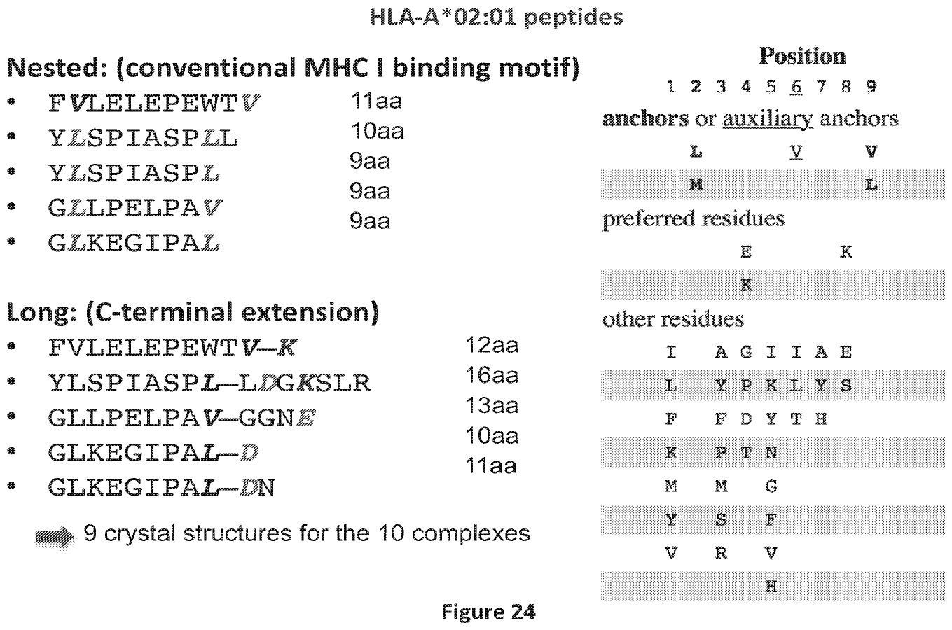

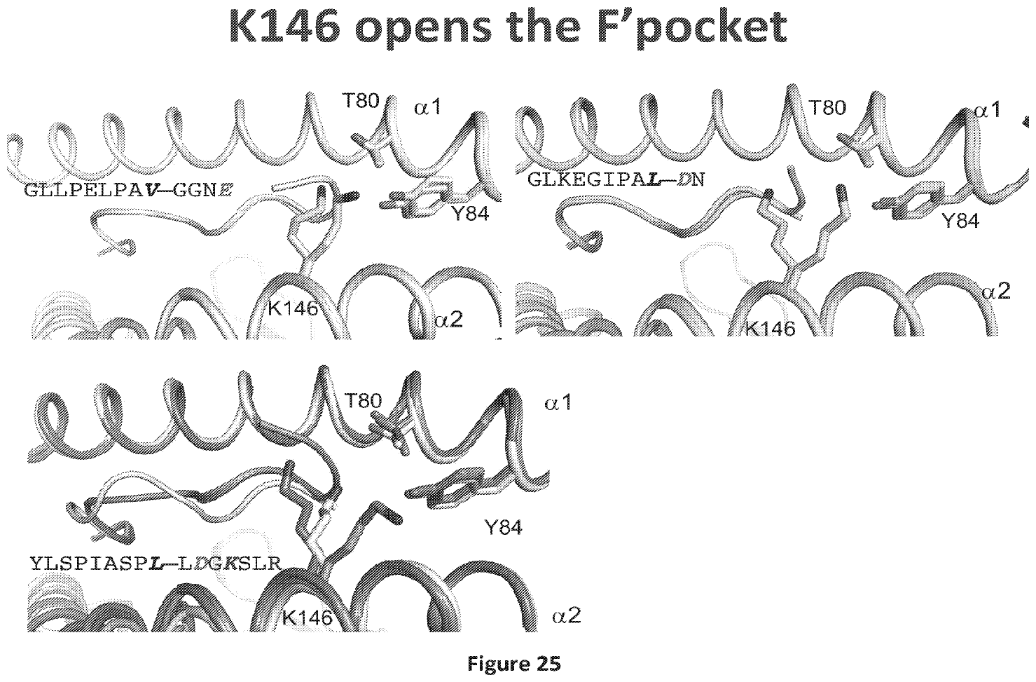

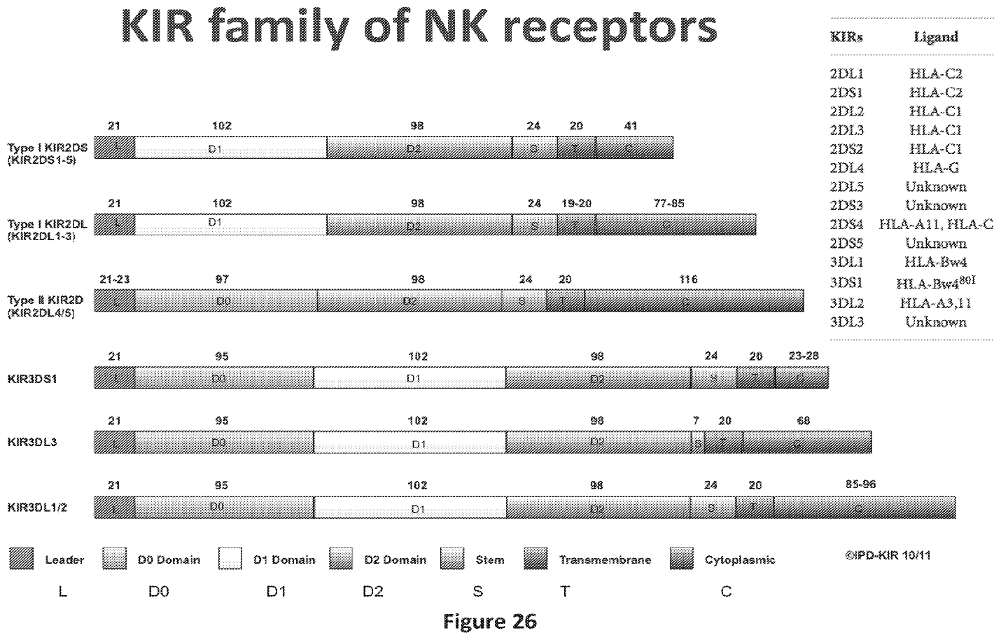

Cytotoxicity assay using KIR transfected YTS cells (FIG. 21) has been established. Peptides (100 μM final concentration) were added to 1×10⁵ 221-HLA cells in 100 μl medium on 96-well plates for 20-24 hrs at 26.5° C. 0.5 million target cells were labeled with 1 μl BATDA reagent on a 37-degree rotator for 30 min. Effector cells (E) and target cells (T) were mixed at 10:1 ratio (50000 E and 5000 T in one well of a V bottom plate) and incubated at 37° C. for 2-4 hrs. Finally, fluorescence was measured in a Perkin Elmer EnVision™ time-resolved fluorometer. YTS cells are tolerized against HLA-C*04:01 through their interacting with KIR2DL1. However, in the absence of this inhibitory signal, YTS cells expressing allele-mismatched KIR3DL1 kill HLA-C*04:01 transfected 721.221 cells (FIG. 21). In a preliminary study the peptide L12K, which was expected to induce the “lysine 146 lift” is sufficiently bound to HLA-B*58:02 to block KIR3DL1 engagement and induce YTS NK cell killing, while the shorter peptide L10K, which was expected to induce the “tyrosine 84 swing” does not induce any killing.

These studies shed light on the therapeutic potential of using C-terminally extended peptides in activating NK cell killing of cancer cells by combining an epitope from Tumor Infiltrating T Lymphocytes with the presently disclosed NK cell activating C-terminal extensions. Without being limited to any particular theory, the ability of extended peptides to inhibit KIR binding likely depends on the efficiency of binding to HLA I molecules expressed on antigen presenting cells. If loading efficiency is low, as determined by the percentage of target cells that were killed, one can start with optimal binding peptides for each HLA I allele that either is predicted or measured to bind with very high affinity. Since peptide binding affinity is not always reduced by the C-terminal extension (10), this increased affinity is expected to favor loading onto empty HLA I molecules on the cell surface

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

### REFERENCES

1. Romagné F, Vivier E. Natural killer cell-based therapies. F1000 medicine reports. 2011; 3:9. doi: 10.3410/M3-9. PubMed PMID: 21654924; PubMed Central PMCID: PMCPMC3096881.
2. Thielens A, Vivier E, Romagné F. N K cell MHC class I specific receptors (KIR): from biology to clinical intervention. Current Opinion in Immunology. 2012; 24(2):239-45. doi: 10.1016/j.coi.2012.01.001. PubMed PMID: 22264929.
3. Vivier E, Ugolini S, Blaise D, Chabannon C, Brossay L. Targeting natural killer cells and natural killer T cells in cancer. Nature reviews Immunology. 2012; 12(4):239-52. doi: 10.1038/nri3174. PubMed PMID: 22437937.
4. Borbulevych O Y, Santhanagopolan S M, Hossain M, Baker B M. TCRs used in cancer gene therapy cross-react with MART-1/Melan-A tumor antigens via distinct mechanisms. Journal of immunology (Baltimore, Md.: 1950). 2011; 187(5):2453-63. doi: 10.4049/jimmunol. 1101268. PubMed PMID: 21795600; PubMed Central PMCID: PMCPMC3166883.
5. Rossjohn J, Gras S, Miles J J, Turner S J, Godfrey D I, McCluskey J. T cell antigen receptor recognition of antigen-presenting molecules. Annu Rev Immunol. 2015; 33:169-200. doi: 10.1146/annurev-immunol-032414-112334. PubMed PMID: 25493333.
6. Walker B, McMichael A. The T-cell response to HIV. Cold Spring Harbor perspectives in medicine. 2012; 2(11). doi: 10.1101/cshperspect.a007054. PubMed PMID: 23002014; PubMed Central PMCID: PMC3543107.
7. Waggoner S N, Reighard S D, Gyurova I E, Cranert S A, Mahl S E, Karmele E P, McNally J P, Moran M T, Brooks T R, Yaqoob F, Rydyznski C E. Roles of natural killer cells in antiviral immunity. Current opinion in virology. 2015; 16:15-23. doi: 10.1016/j.coviro.2015.10.008. PubMed PMID: 26590692.
8. Benson D M, Bakan C E, Zhang S, Collins S M, Liang J, Srivastava S, Hofmeister C C, Efebera Y, André P, Romagné F, Bléry M, Bonnafous C, Zhang J, Clever D, Caligiuri M A, Farag S S. IPH2101, a novel anti-inhibitory KIR antibody, and lenalidomide combine to enhance the natural killer cell versus multiple myeloma effect. Blood. 2011; 118(24):6387-91. doi: 10.1182/blood-2011-06-360255. PubMed PMID: 22031859; PubMed Central PMCID: PMCPMC3490103.
9. Romagné F, André P, Spee P, Zahn S, Anfossi N, Gauthier L, Capanni M, Ruggeri L, Benson D M, Blaser B W, Della Chiesa M, Moretta A, Vivier E, Caligiuri M A, Velardi A, Wagtmann N. Preclinical characterization of 1-7F9, a novel human anti-KIR receptor therapeutic antibody that augments natural killer-mediated killing of tumor cells. Blood. 2009; 114(13):2667-77. doi: 10.1182/blood-2009-02-206532. PubMed PMID: 19553639; PubMed Central PMCID: PMCPMC2756126.
10. McMurtrey C, Trolle T, Sansom T, Remesh S G, Kaever T, Bardet W, Jackson K, Nielsen M, McLeod R, Zajonc D M, Blader I J, Peters B, Sette A, Hildebrand W. peptide ligands open the gate of the HLA class I binding groove. eLife. 2016; 5. doi: 10.7554/eLife.12556. PubMed PMID: 26824387.
11. Remesh S G, Andreatta M, Ying G, Kaever T, Nielsen M, McMurtrey C, Hildebrand W, Peters B, Zajonc D M. Unconventional Peptide Presentation by Major Histocompatibility Complex (MHC) Class I Allele HLA-A*02:01: BREAKING CONFINEMENT. J Biol Chem. 2017; 292(13):5262-70. doi: 10.1074/jbc.M117.776542. PubMed PMID: 28179428; PubMed Central PMCID: PMCPMC5392673.
12. Adams E J, Luoma A M. The adaptable major histocompatibility complex (MHC) fold: structure and function of nonclassical and MHC class I-like molecules. Annu Rev Immunol. 2013; 31:529-61. doi: 10.1146/annurev-immunol-032712-095912. PubMed PMID: 23298204.
13. Rudolph M G, Stanfield R L, Wilson I A. How TCRs bind MHCs, peptides, and coreceptors. Annu Rev Immunol. 2006; 24:419-66. PubMed PMID: 16411852.
14. Madden D R. The three-dimensional structure of peptide-MHC complexes. Annu Rev Immunol. 1995; 13:587-622.
15. Fremont D H, Matsumura M, Stura E A, Peterson P A, Wilson I A. Crystal structures of two viral peptides in complex with murine MHC class I H-2Kb. Science. 1992; 257(5072):919-27. PubMed PMID: 1323877.
16. Guo H C, Jardetzky T S, Garrett T P, Lane W S, Strominger J L, Wiley D C. Different length peptides bind to HLA-Aw68 similarly at their ends but bulge out in the middle. Nature. 1992; 360(6402):364-6. doi: 10.1038/360364a0. PubMed PMID: 1448153.
17. Zhang W, Young A C, Imarai M, Nathenson S G, Sacchettini J C. Crystal structure of the major histocompatibility complex class I H-2Kb molecule containing a single viral peptide: implications for peptide binding and T-cell receptor recognition. Proc Natl Acad Sci USA. 1992; 89(17):8403-7. PubMed PMID: 1325657; PubMed Central PMCID: PMC49927.
18. Hoof I, Peters B, Sidney J, Pedersen L E, Sette A, Lund O, Buus S, Nielsen M. NetMHCpan, a method for MHC class I binding prediction beyond humans. Immunogenetics. 2009; 61(1):1-13. doi: 10.1007/s00251-008-0341-z. PubMed PMID: 19002680; PubMed Central PMCID: PMC3319061.
19. Nielsen M, Lundegaard C, Blicher T, Lamberth K, Harndahl M, Justesen S, Roder G, Peters B, Sette A, Lund O, Buus S. NetMHCpan, a method for quantitative predictions of peptide binding to any HLA-A and -B locus protein of known sequence. PloS one. 2007; 2(8):e796. doi: 10.1371/journal.pone.0000796. PubMed PMID: 17726526; PubMed Central PMCID: PMC1949492.
20. Wang P, Sidney J, Dow C, Mothe B, Sette A, Peters B. A systematic assessment of MHC class II peptide binding predictions and evaluation of a consensus approach. PLoS computational biology. 2008; 4(4):e1000048. doi: 10.1371/journal.pcbi.1000048. PubMed PMID: 18389056; PubMed Central PMCID: PMC2267221.
21. Wang P, Sidney J, Kim Y, Sette A, Lund O, Nielsen M, Peters B. Peptide binding predictions for HLA DR, DP and DQ molecules. BMC bioinformatics. 2010; 11:568. doi: 10.1186/1471-2105-11-568. PubMed PMID: 21092157; PubMed Central PMCID: PMC2998531.
22. Heath W R, Carbone F R. Cross-presentation in viral immunity and self-tolerance. Nature reviews Immunology. 2001; 1(2):126-34. doi: 10.1038/35100512. PubMed PMID: 11905820.
23. Heath W R, Carbone F R. Cross-presentation, dendritic cells, tolerance and immunity. Annu Rev Immunol. 2001; 19:47-64. doi: 10.1146/annurev.immunol.19.1.47. PubMed PMID: 11244030.
24. Vivier E, Raulet D H, Moretta A, Caligiuri M A, Zitvogel L, Lanier L L, Yokoyama W M, Ugolini S. Innate or adaptive immunity? The example of natural killer cells. Science. 2011; 331(6013):44-9. doi: 10.1126/science.1198687. PubMed PMID: 21212348; PubMed Central PMCID: PMC3089969.
25. Verweij M C, Horst D, Griffin B D, Luteijn R D, Davison A J, Ressing M E, Wiertz E J. Viral inhibition of the transporter associated with antigen processing (TAP): a striking example of functional convergent evolution. PLoS pathogens. 2015; 11(4):e1004743. doi: 10.1371/journal.ppat.1004743. PubMed PMID: 25880312; PubMed Central PMCID: PMC4399834.
26. Piguet V. Receptor modulation in viral replication: HIV, HSV, HHV-8 and HPV: same goal, different techniques to interfere with MHC-I antigen presentation. Current topics in microbiology and immunology. 2005; 285:199-217. PubMed PMID: 15609505.
27. Hegde N R, Chevalier M S, Johnson D C. Viral inhibition of MHC class II antigen presentation. Trends in immunology. 2003; 24(5):278-85. PubMed PMID: 12738423.
28. Fruh K, Ahn K, Peterson P A. Inhibition of MHC class I antigen presentation by viral proteins. Journal of molecular medicine. 1997; 75(1):18-27. PubMed PMID: 9020380.
29. Sutherland C L, Chalupny N J, Cosman D. The UL16-binding proteins, a novel family of MHC class I-related ligands for NKG2D, activate natural killer cell functions. Immunol Rev. 2001; 181:185-92. PubMed PMID: 11513139.
30. Vivier E, Anfossi N. Inhibitory NK-cell receptors on T cells: witness of the past, actors of the future. Nature reviews Immunology. 2004; 4(3):190-8. doi: 10.1038/nri1306. PubMed PMID: 15039756.
31. Parham P. MHC class I molecules and KIRs in human history, health and survival. Nature reviews Immunology. 2005; 5(3):201-14. doi: 10.1038/nri1570. PubMed PMID: 15719024.
32. Alter G, Martin M P, Teigen N, Carr W H, Suscovich T J, Schneidewind A, Streeck H, Waring M, Meier A, Brander C, Lifson J D, Allen™, Carrington M, Altfeld M. Differential natural killer cell-mediated inhibition of HIV-1 replication based on distinct KIR/HLA subtypes. J Exp Med. 2007; 204(12):3027-36. doi: 10.1084/jem.20070695. PubMed PMID: 18025129; PubMed Central PMCID: PMC2118524.
33. Alter G, Rihn S, Walter K, Nolting A, Martin M, Rosenberg E S, Miller J S, Carrington M, Altfeld M. HLA class I subtype-dependent expansion of KIR3DS1+ and KIR3DL1+ NK cells during acute human immunodeficiency virus type 1 infection. J Virol. 2009; 83(13):6798-805. doi: 10.1128/JVI.00256-09. PubMed PMID: 19386717; PubMed Central PMCID: PMC2698561.
34. Carrington M, Martin M P, van Bergen J. KIR-HLA intercourse in HIV disease. Trends in microbiology. 2008; 16(12):620-7. doi: 10.1016/j.tim.2008.09.002. PubMed PMID: 18976921; PubMed Central PMCID: PMC3463869.
35. Khakoo S I, Thio C L, Martin M P, Brooks C R, Gao X, Astemborski J, Cheng J, Goedert J J, Vlahov D, Hilgartner M, Cox S, Little A M, Alexander G J, Cramp M E, O'Brien S J, Rosenberg W M, Thomas D L, Carrington M. HLA and N K cell inhibitory receptor genes in resolving hepatitis C virus infection. Science. 2004; 305(5685):872-4. doi: 10.1126/science.1097670. PubMed PMID: 15297676.
36. Vidal-Castineira J R, Lopez-Vazquez A, Diaz-Pena R, Alonso-Arias R, Martinez-Borra J, Perez R, Fernandez-Suarez J, Melon S, Prieto J, Rodrigo L, Lopez-Larrea C. Effect of killer immunoglobulin-like receptors in the response to combined treatment in patients with chronic hepatitis C virus infection. J Virol. 2010; 84(1):475-81. doi: 10.1128/JVI.01285-09. PubMed PMID: 19846535; PubMed Central PMCID: PMC2798445.
37. Ahlenstiel G, Martin M P, Gao X, Carrington M, Rehermann B. Distinct KIR/HLA compound genotypes affect the kinetics of human antiviral natural killer cell responses. The Journal of clinical investigation. 2008; 118(3):1017-26. doi: 10.1172/JCI32400. PubMed PMID: 18246204; PubMed Central PMCID: PMC2214845.
38. Moesta A K, Norman P J, Yawata M, Yawata N, Gleimer M, Parham P. Synergistic polymorphism at two positions distal to the ligand-binding site makes KIR2DL2 a stronger receptor for HLA-C than KIR2DL3. J Immunol. 2008; 180(6):3969-79. PubMed PMID: 18322206.
39. Terme M, Ullrich E, Delahaye N F, Chaput N, Zitvogel L. Natural killer cell-directed therapies: moving from unexpected results to successful strategies. Nat Immunol. 2008; 9(5):486-94. doi: 10.1038/ni1580. PubMed PMID: 18425105.
40. Coca S, Perez-Piqueras J, Martinez D, Colmenarejo A, Saez M A, Vallejo C, Martos J A, Moreno M. The prognostic significance of intratumoral natural killer cells in patients with colorectal carcinoma. Cancer. 1997; 79(12):2320-8. PubMed PMID: 9191519.
41. Chang C C, Campoli M, Ferrone S. Classical and nonclassical HLA class I antigen and N K Cell-activating ligand changes in malignant cells: current challenges and future directions. Advances in cancer research. 2005; 93:189-234. doi: 10.1016/S0065-230X(05)93006-6. PubMed PMID: 15797448.
42. Gasser S, Orsulic S, Brown E J, Raulet D H. The DNA damage pathway regulates innate immune system ligands of the NKG2D receptor. Nature. 2005; 436(7054):1186-90. doi: 10.1038/nature03884. PubMed PMID: 15995699; PubMed Central PMCID: PMC1352168.
43. Johansson S E, Hejdeman B, Hinkula J, Johansson M H, Romagné F, Wahren B, Wagtmann N R, Kirre K, Berg L. N K cell activation by KIR-binding antibody 1-7F9 and response to HIV-infected autologous cells in viremic and controller HIV-infected patients. Clinical immunology (Orlando, Fla.). 2010; 134(2):158-68. doi: 10.1016/j.clim.2009.10.001. PubMed PMID: 19880352.
44. Horig H, Young A C, Papadopoulos N J, DiLorenzo T P, Nathenson S G. Binding of longer peptides to the H-2Kb heterodimer is restricted to peptides extended at their C terminus: refinement of the inherent MHC class I peptide binding criteria. J Immunol. 1999; 163(8):4434-41. PubMed PMID: 10510385.
45. Collins E J, Garboczi D N, Wiley D C. Three-dimensional structure of a peptide extending from one end of a class I MHC binding site. Nature. 1994; 371(6498):626-9. doi: 10.1038/371626a0. PubMed PMID: 7935798.
46. Vivian J P, Duncan R C, Berry R, O&apos; Connor G M, Reid H H, Beddoe T, Gras S, Saunders P M, Olshina M A, Widjaja J M L, Harpur C M, Lin J, Maloveste S M, Price D A, Lafont B A P, McVicar D W, Clements C S, Brooks A G, Rossjohn J. Killer cell immunoglobulin-like receptor 3DL1-mediated recognition of human leukocyte antigen B. Nature. 2011; 479(7373):401-5. doi: 10.1038/nature10517. PubMed PMID: 22020283; PubMed Central PMCID: PMCPMC3723390.
47. Boyington J C, Motyka S A, Schuck P, Brooks A G, Sun P D. Crystal structure of an N K cell immunoglobulin-like receptor in complex with its class I MHC ligand. Nature. 2000; 405(6786):537-43. doi: 10.1038/35014520. PubMed PMID: 10850706.
48. Fan Q R, Long E O, Wiley D C. Crystal structure of the human natural killer cell inhibitory receptor KIR2DL1-HLA-Cw4 complex. Nature Immunology. 2001; 2(5):452-60. doi: 10.1038/87766. PubMed PMID: 11323700.
49. Pymm P, Illing P T, Ramarathinam S H, O'Connor G M, Hughes V A, Hitchen C, Price D A, Ho B K, McVicar D W, Brooks A G, Purcell A W, Rossjohn J, Vivian J P. MHC-I peptides get out of the groove and enable a novel mechanism of HIV-1 escape. Nat Struct Mol Biol. 2017; 24(4):387-94. doi: 10.1038/nsmb.3381. PubMed PMID: 28218747.
50. Buchsbaum S, Barnea E, Dassau L, Beer I, Milner E, Admon A. Large-scale analysis of HLA peptides presented by HLA-Cw4. Immunogenetics. 2003; 55(3):172-6. doi: 10.1007/s00251-003-0570-0. PubMed PMID: 12750860.
51. O'Connor G M, Vivian J P, Gostick E, Pymm P, Lafont B A, Price D A, Rossjohn J, Brooks A G, McVicar D W. Peptide-Dependent Recognition of HLA-B*57:01 by KIR3DS1. J Virol. 2015; 89(10):5213-21. doi: 10.1128/JVI.03586-14. PubMed PMID: 25740999; PubMed Central PMCID: PMCPMC4442525.
52. Li Y, Girardi E, Wang J, Yu E D, Painter G F, Kronenberg M, Zajonc D M. The Vα14 invariant natural killer T cell TCR forces microbial glycolipids and CD1d into a conserved binding mode. J Exp Med. 2010; 207(11):2383-93. Epub 2010/10/06. doi: jem.20101335 [pii]10.1084/jem.20101335. PubMed PMID: 20921281; PubMed Central PMCID: PMC2964572.
53. Aspeslagh S, Li Y, Yu E D, Pauwels N, Trappeniers M, Girardi E, Decruy T, Van Beneden K, Venken K, Drennan M, Leybaert L, Wang J, Franck R W, Van Calenbergh S, Zajonc D M, Elewaut D. Galactose-modified iNKT cell agonists stabilized by an induced fit of CD1d prevent tumour metastasis. Embo J. 2011; 30(11):2294-305. Epub 2011/05/10. doi: emboj2011145 [pii]10.1038/emboj.2011.145. PubMed PMID: 21552205.
54. Girardi E, Yu E D, Li Y, Tarumoto N, Pei B, Wang J, Illarionov P, Kinjo Y, Kronenberg M, Zajonc D M. Unique interplay between sugar and lipid in determining the antigenic potency of bacterial antigens for NKT cells. PLoS biology. 2011; 9(11):e1001189. Epub 2011/11/10. doi: 10.1371/journal.pbio. 1001189. PubMed PMID: 22069376; PubMed Central PMCID: PMC3206013.
55. Yu E D, Girardi E, Wang J, Zajonc D M. Cutting Edge: Structural Basis for the Recognition of {beta}-Linked Glycolipid Antigens by Invariant NKT Cells. J Immunol. 2011; 187(5):2079-83. Epub 2011/08/04. doi: jimmunol.1101636 [pii]10.4049/jimmunol. 1101636. PubMed PMID: 21810611; PubMed Central PMCID: PMC3159715.
56. Girardi E, Maricic I, Wang J, Mac T T, Iyer P, Kumar V, Zajonc D M. Type II natural killer T cells use features of both innate-like and conventional T cells to recognize sulfatide self antigens. Nature immunology. 2012; 13(9):851-6. Epub 2012/07/24. doi: 10.1038/ni.2371. PubMed PMID: 22820602; PubMed Central PMCID: PMC3442777.
57. Aspeslagh S, Nemcovic M, Pauwels N, Venken K, Wang J, Van Calenbergh S, Zajonc D M, Elewaut D. Enhanced TCR footprint by a novel glycolipid increases NKT-dependent tumor protection. J Immunol. 2013; 191(6):2916-25. doi: 10.4049/jimmunol. 1203134. PubMed PMID: 23960235; PubMed Central PMCID: PMC3817951.
58. Betser-Cohen G, Mizrahi S, Elboim M, Alsheich-Bartok O, Mandelboim O. The association of MHC class I proteins with the 2B4 receptor inhibits self-killing of human N K cells. J Immunol. 2010; 184(6):2761-8. doi: 10.4049/jimmunol.0901572. PubMed PMID: 20164429.
59. Colantonio A D, Bimber B N, Neidermyer W J, Jr., Reeves R K, Alter G, Altfeld M, Johnson R P, Carrington M, O'Connor D H, Evans D T. KIR polymorphisms modulate peptide-dependent binding to an MHC class I ligand with a Bw6 motif. PLoS pathogens. 2011; 7(3):e1001316. doi: 10.1371/journal.ppat. 1001316. PubMed PMID: 21423672; PubMed Central PMCID: PMCPMC3053351.
60. Bossi G, Gerry A B, Paston S J, Sutton D H, Hassan N J, Jakobsen B K. Examining the presentation of tumor-associated antigens on peptide-pulsed T2 cells. Oncoimmunology. 2013; 2(11):e26840. doi: 10.4161/onci.26840. PubMed PMID: 24482751; PubMed Central PMCID: PMCPMC3894244.
61. Purbhoo M A, Sutton D H, Brewer J E, Mullings R E, Hill M E, Mahon™, Karbach J, Jager E, Cameron B J, Lissin N, Vyas P, Chen J L, Cerundolo V, Jakobsen B K. Quantifying and imaging N Y-ESO-1/LAGE-1-derived epitopes on tumor cells using high affinity T cell receptors. J Immunol. 2006; 176(12):7308-16. PubMed PMID: 16751374.
62. Peggs K, Verfuerth S, Pizzey A, Ainsworth J, Moss P, Mackinnon S. Characterization of human cytomegalovirus peptide-specific CD8(+) T-cell repertoire diversity following in vitro restimulation by antigen-pulsed dendritic cells. Blood. 2002; 99(1):213-23. PubMed PMID: 11756174.
63. Wills M R, Carmichael A J, Mynard K, Jin X, Weekes M P, Plachter B, Sissons J G. The human cytotoxic T-lymphocyte (CTL) response to cytomegalovirus is dominated by structural protein pp65: frequency, specificity, and T-cell receptor usage of pp65-specific CTL. J Virol. 1996; 70(11):7569-79. PubMed PMID: 8892876; PubMed Central PMCID: PMCPMC190825.
64. McLaughlin-Taylor E, Pande H, Forman S J, Tanamachi B, Li C R, Zaia J A, Greenberg P D, Riddell S R. Identification of the major late human cytomegalovirus matrix protein pp65 as a target antigen for CD8+ virus-specific cytotoxic T lymphocytes. J Med Virol. 1994; 43(1):103-10. PubMed PMID: 8083644.
65. Burshtyn D N, Shin J, Stebbins C, Long E O. Adhesion to target cells is disrupted by the killer cell inhibitory receptor. Current biology: C B. 2000; 10(13):777-80. PubMed PMID: 10898979.
66. Saunders P M, Vivian J P, Baschuk N, Beddoe T, Widjaja J, O&apos; Connor G M, Hitchen C, Pymm P, Andrews D M, Gras S, McVicar D W, Rossjohn J, Brooks A G. The interaction of KIR3DL1*001 with HLA class I molecules is dependent upon molecular microarchitecture within the Bw4 epitope. Journal of immunology (Baltimore, Md.: 1950). 2015; 194(2):781-9. doi: 10.4049/jimmunol.1402542. PubMed PMID: 25480565; PubMed Central PMCID: PMCPMC4282956.
67. Lecoeur H, Février M, Garcia S, Rivière Y, Gougeon M L. A novel flow cytometric assay for quantitation and multiparametric characterization of cell-mediated cytotoxicity. Journal of Immunological Methods. 2001; 253(1-2): 177-87. PubMed PMID: 11384679.
68. Iversen A C, Norris P S, Ware C F, Benedict C A. Human N K cells inhibit cytomegalovirus replication through a noncytolytic mechanism involving lymphotoxin-dependent induction of IFN-beta. J Immunol. 2005; 175(11):7568-74. PubMed PMID: 16301666.
69. Sedy J R, Bjordahl R L, Bekiaris V, Macauley M G, Ware B C, Norris P S, Lurain N S, Benedict C A, Ware C F. CD160 activation by herpesvirus entry mediator augments inflammatory cytokine production and cytolytic function by N K cells. J Immunol. 2013; 191(2):828-36. doi: 10.4049/jimmunol.1300894. PubMed PMID: 23761635; PubMed Central PMCID: PMC3702646.
70. Sola C, André P, Lemmers C, Fuseri N, Bonnafous C, Bléry M, Wagtmann N R, Romagné F, Vivier E, Ugolini S. Genetic and antibody-mediated reprogramming of natural killer cell missing-self recognition in vivo. Proceedings of the National Academy of Sciences of the United States of America. 2009; 106(31):12879-84. doi: 10.1073/pnas.0901653106. PubMed PMID: 19561305; PubMed Central PMCID: PMCPMC2722344.

What is claimed is:

1. A synthetic polypeptide comprising:
(a) a first peptide sequence consisting of 7 to 11 amino acids in length and comprising an MHC class I binding epitope; and
(b) no more than one additional amino acid covalently linked to a C-terminus of the first peptide, wherein the one additional amino acid comprises an electronegative or electropositive charged side group, or a second peptide consisting of 2 to 7 amino acids in length covalently linked to a C-terminus of the first peptide, wherein the second peptide comprises at least one electronegative or electropositive charged side group.

2. The synthetic polypeptide of claim 1, wherein the MHC class I binding epitope comprises an HLA-A, HLA-B or HLA-C epitope.

3. The synthetic polypeptide of claim 1 or 2, wherein the HLA-A epitope comprises the amino acid sequence X₁X₂X₃X₄X₈X₆X₇X₈X₉, wherein X₁ is F, Y, G, I, L, K, M or V; X₂ is L, M or V; X₃ is L, S, K, A, Y, F, P, M, or R; X₄ is E, P, K, G, D or T; X₅ is I, L, E, G, K, Y, N, F, V or H; X₆ is E, A, L, I, V or T; X₇ is P, S, A, Y or H; X₈ is E, P, A, K, or S; and X₉ is W, L, or V.

4. The synthetic polypeptide of claim 3, wherein the HLA-A epitope comprises the amino acid sequence FVLELEPEWT, FVLELEPEWTV, YLSPIASPL, YLSPIASPLL, GLLPELPAV, GLKEGIPAL, or AAFIFILTV.

5. The synthetic polypeptide of claim 2, wherein the HLA-B epitope comprises the amino acid sequence LSSPVTKSF.

6. The synthetic polypeptide of claim 2, wherein the HLA-C epitope comprises the amino acid sequence GAVDPLLAL.

7. The synthetic polypeptide of any one of claims 1 to 6, wherein the MHC class I binding peptide comprises a non-classical HLA epitope.

8. The synthetic polypeptide of any one of claims 1 to 7, wherein the MHC class I binding epitope comprises an HLA-E or HLA-G epitope.

9. The synthetic polypeptide of any one of claims 1 to 8, wherein the first peptide sequence comprises amino acids that are conserved among HLA-A, HLA-B and HLA-C epitopes.

10. The synthetic polypeptide of any one of claims 1 to 9, wherein the first peptide sequence is 9, 10 or 11 amino acids in length.

11. The synthetic polypeptide of any one of claims 1 to 10, wherein the additional amino acid or second peptide sequence is covalently linked to the C-terminus of the first amino acid sequence by a peptide bond.

12. The synthetic polypeptide of any one of claims 1 to 11, wherein the second peptide sequence is 2, 3, 4, 5, 6, or 7 amino acids in length.

13. The synthetic polypeptide of any one of claims 1 to 11, wherein the second peptide sequence is not more than 3 amino acids in length.

14. The synthetic polypeptide of any one of claims 1 to 13, wherein the second peptide sequence comprises glycine or serine.

15. The synthetic polypeptide of any one of claims 1 to 14, wherein the second peptide sequence comprises valine, alanine or leucine.

16. The synthetic polypeptide of any one of claims 1 to 13, wherein the additional amino acid is glutamate, aspartic acid, or a derivative or variant thereof.

17. The synthetic polypeptide of any one of claims 1 to 13, wherein the additional amino acid is lysine.

18. The synthetic polypeptide of any one of claims 1 to 12, wherein the second peptide sequence is selected from the group consisting of D, K, DN, DG, GGNE, DGK, DGKSLR,

19. The synthetic polypeptide of any one of claims 1 to 18, wherein the synthetic polypeptide is 8 to 18 amino acids in length.

20. The synthetic polypeptide of any one of claims 1 to 19, wherein the synthetic polypeptide is not more than 10, not more than 11, not more than 12, not more than 13, or not more than 14 amino acids in length.

21. The synthetic polypeptide of any one of claims 1 to 20, wherein the synthetic polypeptide binds specifically to an MHC class I molecule.

22. The synthetic polypeptide of claim 21, wherein upon binding, the synthetic polypeptide induces a structural change in the MHC class I molecule, and the structural change comprises opening an F pocket of the MHC class I molecule.

23. The synthetic polypeptide of claim 21 or 22, wherein the binding blocks, abrogates, reduces, decreases or inhibits binding of a killer immunoglobulin receptor (KIR) to the MHC class I molecule.

24. The synthetic polypeptide of claim 23, wherein the KIR is an inhibitor KIR.

25. The synthetic polypeptide of claim 23 or 24, wherein the KIR is expressed on an NK cell.

26. The synthetic polypeptide of any one of claims 1 to 25, wherein at least one electronegative charged side group comprises a carboxyl group.

27. The synthetic polypeptide of any one of claims 1 to 26, wherein at least one electropositive charged side group comprises a primary or secondary amine.

28. The synthetic polypeptide of any one of claims 1 to 27, wherein the synthetic polypeptide comprises or consists of an amino acid sequence selected from the group consisting of AAGIGILTV, AAGIGILTVK, AAGIGILTVD, AAGIGILTVDGK, TNLVPMVATV, TNLVPMVATVDGK, LSSPVTKSF, LSSPVTKSFK, LSSPVTKSFD, LSSPVTKSFDGK, GAVDPLLAL, GAVDPLLALK, GAVDPLLALD, GAVDPLLALDGK, YFDPANGKF, YFDPANGKFK and YFDPANGKFDN.

29. A macromolecule comprising one or more of the synthetic polypeptides of any one of claims 1 to 28.

30. A composition comprising the synthetic polypeptide of any one of claims 1 to 28, or the macromolecule claim 29.

31. The composition of claim 30, wherein the composition is a pharmaceutical composition.

32. The composition of claim 30 or 31, wherein the composition is configured for administration to a subject.

33. A method of treating a subject having a neoplasia, neoplastic disorder or cancer comprising:
a) providing a subject having, or suspected of having, a neoplastic disorder; and
b) administering a therapeutically effective amount of the synthetic polypeptide of any one of claims 1 to 28 to the subject.

34. A method of treating a subject having a neoplasia, neoplastic disorder or cancer comprising:
a) providing a subject having, or suspected of having, a neoplastic disorder; and
b) administering a therapeutically effective amount of the macromolecule, compound or composition of any one of claims 29 to 32 to the subject.

35. A method of reducing or inhibiting proliferation of a neoplastic cell, tumor, cancer or malignant cell, comprising contacting the cell, tumor, cancer or malignant cell, with the synthetic polypeptide of any one of claims 1 to 28 in an amount sufficient to reduce or inhibit proliferation of the neoplastic cell, tumor, cancer or malignant cell.

36. A method of reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy, comprising administering to the subject an amount of the synthetic polypeptide of any one of claims 1 to 28 sufficient to reduce or inhibit metastasis of the neoplasia, tumor, cancer or malignancy to other sites, or formation or establishment of metastatic neoplasia, tumor, cancer or malignancy at other sites distal from the primary neoplasia, tumor, cancer or malignancy.

37. The method of any one of claims 33 to 36, wherein the wherein the neoplasia, neoplastic disorder, tumor, cancer or malignancy comprises a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

38. The method of any one of claims 33 to 37, wherein the neoplasia, neoplastic disorder, tumor, cancer or malignancy comprises hematopoietic cells.

39. The method of any one of claims 37 to 38, wherein the sarcoma comprises a lymphosarcoma, liposarcoma, osteosarcoma, chondrosarcoma, leiomyosarcoma, rhabdomyosarcoma or fibrosarcoma.

40. The method of any one of claims 33 to 38, wherein the neoplasia, neoplastic disorder, tumor, cancer or malignancy comprises a myeloma, lymphoma or leukemia.

41. The method of any one of claims 33 to 38, wherein the neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer.

42. The method of claim 41, wherein the lung neoplasia, neoplastic disorder, tumor, cancer or malignancy comprises small cell lung or non-small cell lung cancer.

43. The method of claims 41 or 42, wherein the neoplasia, neoplastic disorder, tumor, cancer or malignancy comprises a stem cell neoplasia, tumor, cancer or malignancy.

44. The method of any one of claims 33 to 43, wherein the method inhibits, or reduces relapse or progression of the neoplasia, neoplastic disorder, tumor, cancer or malignancy.

45. The method of any one of claims 33 to 44, further comprising administering an anti-cell proliferative, anti-neoplastic, anti-tumor, anti-cancer or immune-enhancing treatment or therapy.

46. The method of any one of claims 33 to 45, wherein the treatment results in partial or complete destruction of the neoplastic, tumor, cancer or malignant cell mass; a reduction in volume, size or numbers of cells of the neoplastic, tumor, cancer or malignant cell mass; stimulating, inducing or increasing neoplastic, tumor, cancer or malignant cell necrosis, lysis or apoptosis; reducing neoplasia, tumor, cancer or malignancy cell mass; inhibiting or preventing progression or an increase in neoplasia, tumor, cancer or malignancy volume, mass, size or cell numbers; or prolonging lifespan.

47. The method of any one of claims 33 to 46, wherein the treatment results in reducing or decreasing severity, duration or frequency of an adverse symptom or complication associated with or caused by the neoplasia, tumor, cancer or malignancy.

48. The method of any one of claims 33 to 47, wherein the treatment results in reducing or decreasing pain, discomfort, nausea, weakness or lethargy.

49. The method of any one of claims 33 to 48, wherein the treatment results in increased energy, appetite, improved mobility or psychological well-being.

50. A method of treating a subject having an infection or infectious disease comprising:
a) providing a subject having, or suspected of having, an infectious disease; and
b) administering a therapeutically effective amount of the synthetic polypeptide of any one of claims 1 to 28 to the subject.

51. A method of treating a subject having an infection or infectious disease comprising:
a) providing a subject having, or suspected of having, an infectious disease; and
b) administering a therapeutically effective amount of the macromolecule, compound or composition of any one of claims 29 to 32 to the subject.

52. A method of reducing or inhibiting proliferation of a virus, bacteria or parasite, comprising contacting the cell with the synthetic polypeptide of any one of claims 1 to 28 in an amount sufficient to reduce or inhibit proliferation of the virus, bacteria or parasite.

53. The method of claim 52, wherein the parasite is T. gondii.

54. The method of claim 52, wherein the bacteria is M. tuberculosis.

55. A method of modulating activity of a Natural Killer cell, the method comprising contacting the NK cell with the synthetic polypeptide of any one of claims 1 to 28.

56. A method of modulating activity of a Natural Killer cell, the method comprising contacting the NK cell with the macromolecule, compound or composition of any one of claims 29 to 32.

57. The method of claim 55 or claim 56, wherein the method comprises activating NK cell activity.

58. The method of any one of claims 55 to 57, wherein the method comprises modulating activity of the Natural Killer cell in a subject.

59. The method of claim 58, wherein the subject has neoplasia, neoplastic disorder or cancer.

60. The method of claim 59, wherein the method comprises reducing or inhibiting proliferation of a neoplastic cell, tumor, cancer or malignant cell in the subject.

61. The method of claim 59, wherein the method comprises reducing or inhibiting metastasis of a neoplasia, tumor, cancer or malignancy to other sites, or formation or establishment of metastatic neoplasia, tumor, cancer or malignancy at other sites distal from a primary neoplasia, tumor, cancer or malignancy in the subject.

62. The method of any one of claims 59 to 61, wherein the wherein the neoplasia, neoplastic disorder, tumor, cancer or malignancy comprises a carcinoma, sarcoma, neuroblastoma, cervical cancer, hepatocellular cancer, mesothelioma, glioblastoma, myeloma, lymphoma, leukemia, adenoma, adenocarcinoma, glioma, glioblastoma, retinoblastoma, astrocytoma, oligodendrocytoma, meningioma, or melanoma.

63. The method of any one of claims 59 to 62, wherein the neoplasia, neoplastic disorder, tumor, cancer or malignancy comprises hematopoietic cells.

64. The method of claim 62 or 63, wherein the sarcoma comprises a lymphosarcoma, liposarcoma, osteosarcoma, chondrosarcoma, leiomyosarcoma, rhabdomyosarcoma or fibrosarcoma.

65. The method of any one of claims 59 to 63, wherein the neoplasia, neoplastic disorder, tumor, cancer or malignancy comprises a myeloma, lymphoma or leukemia.

66. The method of any one of claims 59 to 63, wherein the neoplasia, neoplastic disorder, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, tumor, or cancer.

67. The method of claim 66, wherein the lung neoplasia, neoplastic disorder, tumor, cancer or malignancy comprises small cell lung or non-small cell lung cancer.

68. The method of claims 66 or 67, wherein the neoplasia, neoplastic disorder, tumor, cancer or malignancy comprises a stem cell neoplasia, tumor, cancer or malignancy.

69. The method of any one of claims 59 to 68, wherein the method inhibits, or reduces relapse or progression of the neoplasia, neoplastic disorder, tumor, cancer or malignancy.

70. The method of any one of claims 59 to 69, further comprising administering to the subject an anti-cell proliferative, anti-neoplastic, anti-tumor, anti-cancer or immune-enhancing treatment or therapy.

71. The method of claim 58, wherein the subject has an infection or infectious disease.

72. The method of claim 71, wherein the method comprises reducing or inhibiting proliferation of a virus, bacteria or parasite in the subject.

73. The method of claim 72, wherein the parasite is T. gondii.

74. The method of claim 72, wherein the bacteria is M. tuberculosis.
